(19) United States
(12) Patent Application Publication
(10) Pub. No.: US 2018/0080086 A1
(43) Pub. Date: Mar. 22, 2018

(54) FISH PANEL FOR CTCL

(71) Applicant: Yale University, New Haven, CT (US)

(72) Inventors: Michael Girardi, Madison, CT (US); Jason Weed, Bella Vista, AR (US); Juliet Gibson, Dallas, TX (US); Julia Lewis, Clinton, CT (US)

(73) Assignee: Yale University, New Haven, CT (US)

(21) Appl. No.: 15/688,834

(22) Filed: Aug. 28, 2017

Related U.S. Application Data
(60) Provisional application No. 62/380,849, filed on Aug. 29, 2016.

(51) Int. Cl. C12Q 1/68

(52) U.S. Cl. CPC C12Q 1/6886; C12Q 2600/156

(57) ABSTRACT

Described herein are probes useful to detect gene copy number alterations of genes characteristic/indicative of cutaneous T cell lymphoma (CTCL), such as CTCL with blood involvement, including probes specific to genes amplified or deleted in cutaneous T cell lymphoma (CTCL), such as CTCL with blood involvement; methods of detecting a (one or more) genetic abnormality, such as one or more gene copy number alteration (GCNA), characteristic/indicative of cutaneous T cell lymphoma (CTCL), such as CTCL with blood involvement, in a biological sample obtained from an individual; and methods of determining if an individual has cutaneous T cell lymphoma (CTCL), such as CTCL with blood involvement, or is likely to develop cutaneous T cell lymphoma (CTCL) with blood involvement.

### RELATED APPLICATIONS

This application claims the benefit under 35 U.S.C. § 119(e) of U.S. provisional application No. 62/380,849, filed Aug. 29, 2016, which is incorporated by reference herein in its entirety.

### FEDERALLY SPONSORED RESEARCH

This invention was made with government support under P50 CA121974 awarded by the National Institutes of Health. The government has certain rights in the invention.

### BACKGROUND

At present, no clinical tests are widely used to assess mutational burden in cutaneous T cell lymphoma (CTCL), including CTCL with leukemic involvement (L-CTCL), such as in Sézary syndrome (SS). CTCL cells involve the skin and may develop into various types of skin lesions (such as patches, plaques and tumors), may involve the lymph nodes, and may involve various visceral organs. Aside from the demonstration of T cell clonality by examination of the T cell receptor (TCR) status by Southern hybridization, polymerase chain reaction (PCR), or DNA/RNA sequencing (e.g., by high throughput sequencing (HTS)), there are limited options for molecular testing for CTCL involvement in the blood, skin, lymph node, or visceral organs. Routine blood analysis offers limited guidance for assessing prognosis and optimizing treatment within the highly variable population of affected patients. For now, CTCL contrasts sharply with more common and better-characterized cancers, such as chronic lymphocytic leukemia and ductal carcinoma of the breast, for which mutation analysis stratifies prognostic subgroups and can influence disease management (Nuciforo et al., 2016; Van Dyke et al., 2016).

### SUMMARY

Described herein are probes useful to detect gene copy number alterations of genes characteristic/indicative of cutaneous T cell lymphoma (CTCL), such as CTCL with blood involvement (L-CTCL) or CTCL involving the skin, lymph nodes, or the visceral organs, including probes specific to genes amplified in cutaneous T cell lymphoma (CTCL) and probes specific to genes deleted in cutaneous T cell lymphoma (CTCL), such as CTCL with blood involvement (L-CTCL), including any type of CTCL involving the blood, skin, lymph nodes, and/or the visceral organs; methods of detecting a (one or more) genetic abnormality, such as one or more gene copy number alteration (GCNA), that is characteristic (indicative) of cutaneous T cell lymphoma (CTCL), such as CTCL with blood involvement, including any type of CTCL involving the blood, skin, lymph nodes, and/or the visceral organs, in a biological sample obtained from an individual; and methods of determining if an individual has cutaneous T cell lymphoma (CTCL), such as CTCL with blood involvement, including any type of CTCL involving the blood, skin, lymph nodes, and/or the visceral organs, or is likely to develop cutaneous T cell lymphoma (CTCL) with blood involvement. Specific embodiments are panels of fluorescence in situ hybridization probes specific to genes commonly amplified in CTCL or L-CTCL; panels of fluorescence in situ probes specific to genes commonly deleted in CTCL or L-CTCL; and panels of one or more fluorescence in situ probes specific to genes commonly amplified in CTCL or L-CTCL and one or more fluorescence in situ probes specific to genes commonly deleted in CTCL or L-CTCL. Also described are methods of establishing the likelihood of the presence of (that an individual being assessed has) CTCL or L-CTCL. In specific embodiments of the method, the probes are fluorescence in situ hybridization probes specific to genes (such as those described herein) commonly amplified in CTCL or L-CTCL, or fluorescence in situ hybridization probes specific to genes (such as those described herein) commonly deleted in CTCL or L-CTCL or a combination of one or more fluorescence in situ probes specific to genes commonly amplified in CTCL or L-CTCL and one or more fluorescence in situ probes specific to genes commonly deleted in CTCL or L-CTCL and the method comprises establishing the likelihood of CTCL, including leukemic CTCL (CTCL with blood involvement), including any type of CTCL involving the blood, skin, lymph nodes, and/or the visceral organs, based on results of GCNA analysis.

In one embodiment, the method comprises assessing (enumerating) the copy number of some or all of the biomarkers: (e.g., TP53, MYC, RB1, CDKN2A, ATM, STAT3/5B, ARID1A, ZEB1, FAS, CARD11, and DNMT3A) in a biological sample (e.g., blood, isolated T cells), using a (at least one, one or more) probe for each gene (e.g., probes described herein) and FISH hybridization; determining, for each gene for which copy number was assessed, the portion or percent of cells that show an abnormal copy number; and comparing the portion or percent to a reference value. In one embodiment, if, for any gene, more than about 12% of the population of cells shows an abnormal copy number, the individual from whom the biological sample was obtained is diagnosed as having CTCL, such as CTCL with blood involvement. If no abnormal populations are found, the diagnosis of cutaneous T cell lymphoma with blood involvement is excluded with high likelihood. In a specific embodiment, copy number of each of the 11 genes (TP53, MYC, RB1, CDKN2A, ATM, STAT3/5B, ARID1A, ZEB1, FAS, CARD11, and DNMT3A) is determined in a biological sample (e.g., blood, isolated T cells) obtained from an individual using probes described herein or their equivalents and FISH, and the percent or portion of cells comprising abnormal gene copy number for each gene is determined and compared with a reference value or cutoff. If the percent of cells in which abnormal gene copy is greater than about 12%, the individual is diagnosed as having CTCL or CTCL with blood involvement. For example, the copy number of genes which are frequently amplified or deleted in CTCL (e.g., 11 genes described herein or a subset, such as four, of the 11 genes) is enumerated in abnormal T cell populations obtained from a patient; for example, peripheral blood is drawn, abnormal T cells are enriched (e.g., with flow sorting or magnetic beads) and the sorted population undergoes FISH hybridization with hybridization probes specific to genes commonly amplified or deleted in CTCL. In a specific embodiment, if for any gene assessed, a population over 12% (more than about 12%) of cells shows an abnormal copy number, the patient sample indicates that the patient has cutaneous T cell lymphoma. If no abnormal populations are found, the diagnosis of cutaneous T cell lymphoma is excluded with high likelihood. In another embodiment, copy number of a subset of/fewer than the 11 genes, such as a set of four genes (e.g., TP53, MYC, STAT3/5B, and ARID1A) is determined in a biological sample (e.g., blood, T cells) obtained from an individual, using probes described herein and FISH, and the percent or portion of cells comprising abnormal gene copy number for each gene is determined and compared with a reference value or cutoff. The reference value or cutoff is a percent or portion of cells in a population in which gene copy number alteration (e.g., amplification or deletion) is determined to be indicative of CTCL and particularly, CTCL with blood involvement. For example, the reference value or cutoff has been determined, using methods known to those of skill in the art (e.g., methods described herein), from assessing biological samples from individuals who have CTCL. The percent or portion of cells comprising a gene alteration (gene copy number alteration, such as amplification or deletion) for one or more of the genes assessed (e.g., one or more of TP53, MYC, RB1, CDKN2A, ATM, STAT3/5B, ARID1A, ZEB1, FAS, CARD11, and DNMT3A) in individuals who have CTCL can serve as the reference value or cutoff. For example, if the percent of cells in which abnormal gene copy for at least one of the genes is greater than about 12%, the individual is diagnosed as having CTCL with blood involvement. The reference value or cutoff can be a previously determined number, such as described herein, a later-determined number or a number determined close in time/at the same time as assessment of the biological sample from the individual is carried out.

In the embodiments described, a biological sample is assessed. For example, leukocytes are obtained from patient tissue (e.g. blood, skin, lymph node, visceral organ). Lymphocytes may also be sorted to isolate abnormal T cell populations consistent with cutaneous T cell lymphoma, thereby enriching for CD3+ lymphocytes, CD4+ lymphocytes, and/or CD3+CD4+ lymphocytes. The biological sample may also be enriched for loss of T cell markes, such as CD7− lymphocytes and/or CD26− lymphocytes. These selected cells undergo FISH hybridization to a panel of probes specific for genes commonly amplified or deleted in CTCL or L-CTCL. Abnormal FISH populations are then quantified, and if one or more probes shows abnormal signal number in more than a percent or portion of cells (a cutoff or reference value) visualized, a diagnosis of CTCL or L-CTCL is made. If no abnormal populations are found, the diagnosis of CTCL or L-CTCL is excluded with high likelihood.

The probe panel and diagnostic algorithm described capture genetic abnormalities of malignant cells, in contrast to current diagnostic tools, which rely on 1) measurements of cell surface markers that do not strongly indicate clonality or 2) measurement of T cell receptor clonality which has been demonstrated as suboptimally sensitive and suboptimally specific.

Described herein are methods and probes, such as a panel of probes (e.g., fluorescence in situ hybridization (FISH) probes) useful to identify individuals with cutaneous T cell lymphoma (CTCL) or CTCL with blood involvement (L-CTCL). The probes are useful, as described herein, to capture gene copy number alterations (GCNAs) in individuals (humans) with CTCL or CTCL with blood involvement. Gene copy number alterations in some or all of TP53, MYC, RB1, CDKN2A, ATM, STAT3/5B, ARID1A, ZEB1, FAS, CARD11, and DNMT3A are assessed/determined, using probes described herein or equivalent probes (probes that comprise different sequences but detect alterations, such as GCNAs, detected by the probes described herein). Abnormalities, such as gene copy number alteration, are detected in one or more of TP53, MYC, RB1, CDKN2A, ATM, STAT3/5B, ARID1A, ZEB1, FAS, CARD11, and DNMT3A and, if at least one abnormality (e.g., GCNA) is detected in a sample obtained from an individual (e.g., blood, lymph), the percent or portion of cells in which the at least one abnormality occurs is compared with a reference value (e.g., with the percent or portion of cells in which gene copy number abnormality has been shown to be present in individuals with CTCL or CTCL with blood involvement). If the percent or portion of cells in the biological sample that have abnormal gene copy number is equal to or greater than the reference, the biological sample comprises GCNAs indicative of CTCL or CTCL with blood involvement. One or more probes (2, 3, 4, 5, 6, 7, 8, 9, 10 or 11) can be used to assess copy number of the gene for which it is specific. A probe panel can include any number of probes for some or all of the 11 genes. Described is a panel of 11 probes, such as fluorescence in situ hybridization (FISH) probes, designed to capture gene copy number alterations (GCNAs) in patients with L-CTCL. The panel includes a (at least one) probe for each of TP53, MYC, RB1, CDKN2A, ATM, STAT3/5B, ARID1A, ZEB1, FAS, CARD11, and DNMT3A (FIG. 1, panel A). FISH technology particularly suits CTCL, a cancer whose mutational landscape is characterized by a preponderance of gene copy number amplifications and deletions and not single nucleotide variants relatively overrepresented in many other forms of cancer. The 11-probe panel was used to assess genetic abnormalities in sorted or unsorted peripheral blood from 24 patients (Patient Characteristics, Table 6) with a range of disease presentations, including Sézary syndrome (SS), patch/plaque mycosis fungoides (MF), folliculotropic mycosis fungoides (F-MF), tumor-stage mycosis fungoides, and follicular mucinosis—with evidence of blood involvement (Gibson et al., 2016).

In one embodiment, the method is a method of detecting a genetic abnormality associated with cutaneous T cell lymphoma (CTCL) or CTCL with blood involvement, comprising analyzing a biological sample for a genetic abnormality in TP53, MYC, RB1, CDKN2A, ATM, STAT3/5B, ARID1A, ZEB1, FAS, CARD11, and DNMT3A; and determining the presence or absence of a genetic abnormality in at least one of TP53, MYC, RB1, CDKN2A, ATM, STAT3/5B, ARID1A, ZEB1, FAS, CARD11, and DNMT3A, wherein if there is a genetic abnormality in at least one of TP53, MYC, RB1, CDKN2A, ATM, STAT3/5B, ARID1A, ZEB1, FAS, CARD11, and DNMT3A, a genetic abnormality associated with CTCL or L-CTCL with blood involvement is detected. It is not necessary that the biological sample (cells) be assessed for alteration of all 11 genes; a sample can be assessed for fewer. For example, if a biological sample is determined to have a genetic abnormality (e.g., gene amplification) in one of the 11 genes, that is sufficient to conclude that the individual has CTCL or L-CTCL and additional assessment is not needed. In specific embodiments, if more than about 12% of the population of cells. In some embodiments, analyzing a biological sample comprises analyzing genetic abnormalities in a cell. In some embodiments, cells are obtained from blood. In some embodiments, cells are obtained from tissue (e.g., skin, lymph node, or visceral organs). It is not necessary that the blood be the source of potential CTCL cells for analysis by FISH. Cells in question may also be attainable by isolation from tissues including the skin, lymph node, or visceral organs.

In some embodiments, the present disclosure provides a method of detecting, in a biological sample obtained from an individual, a genetic abnormality associated with cutaneous T cell lymphoma (CTCL) or CTCL with blood involvement, comprising assessing TP53, MYC, RB1, CDKN2A, ATM, STAT3/5B, ARID1A, ZEB1, FAS, CARD11, and DNMT3A for a genetic abnormality, wherein if a genetic abnormality is present in at least one of TP53, MYC, RB1, CDKN2A, ATM, STAT3/5B, ARID1A, ZEB1, FAS, CARD11, and DNMT3A, the biological sample comprises a genetic abnormality associated with CTCL or CTCL with blood involvement.

A further embodiment is a method of detecting cutaneous T cell lymphoma (CTCL) or CTCL with blood involvement, comprising analyzing nucleic acids from a biological sample for a genetic abnormality in TP53, MYC, RB1, CDKN2A, ATM, STAT3/5B, ARID1A, ZEB1, FAS, CARD11, and DNMT3A; and if the biological sample is determined as having one or more genetic abnormalities in TP53, MYC, RB1, CDKN2A, ATM, STAT3/5B, ARID1A, ZEB1, FAS, CARD11, and DNMT3A identifying as a biological sample having CTCL or CTCL with blood involvement, wherein the genetic abnormality comprises at least one deletion in at least one of TP53, RB1, CDKN2A, ATM, ARID1A, ZEB1, FAS, and DNMT3A; at least one amplification in at least one of MYC, STAT3/5B, and CARD11; or at least one deletion in at least one of TP53, RB1, CDKN2A, ATM, ARID1A, ZEB1, FAS, and DNMT3A and at least one amplification in at least one of MYC, STAT3/5B, and CARD11.

In some embodiments, the method of detecting cutaneous T cell lymphoma (CTCL) or CTCL with blood involvement comprises analyzing nucleic acids from the biological sample for a genetic abnormality in one or more of TP53, MYC, STAT3/5B, and ARID1A; and identifying the biological sample having one or more genetic abnormalities in TP53, MYC, STAT3/5B, and ARID1A as a biological sample having CTCL or CTCL with blood involvement, wherein the genetic abnormality comprises at least a single deletion in TP53 or ARID1A, at least a single amplification in MYC or STAT3/5B or at least one deletion in TP53 or ARID1A and at least a single amplification in MYC or STAT3/3B. If the percent of cells in the population in which abnormal gene copy for at least one of the genes is greater than about 12%, the individual is diagnosed as having CTCL or CTCL with blood involvement. A subset of just four probes could be combined into one blood test predicted to be abnormal in over 90% of patients. This would be less expensive than using all the probes. Such a panel could be useful for actually detecting blood involvement of genetically abnormal cells rather than relying on cutoff percentages in flow cytometry or the T cell receptor PCR which exhibit suboptimal sensitivity and very suboptimal specificity.

A genetic abnormality associated with CTCL or CTCL with blood involvement may comprise a deletion or an amplification. In some embodiments, the genetic abnormality comprises a single deletion. In some embodiments, the genetic abnormality comprises a double deletion. In some embodiments, the genetic abnormality comprises a single amplification. In some embodiments, the genetic abnormality comprises a multiple amplification. See, for example, deletions and amplifications in the 11 genes described here, as represented in FIG. 2.

In some embodiments, the deletion or abnormality is detected by combining the biological sample with a probe selected from ARID1A (Probe 1), ARID1A (Probe 2), ARID1A (Probe 3), CARD11 (Probe 1), CARD11 (Probe 2), CARD11 (Probe 3), ZEB1 (Probe 1), ZEB1 (Probe 2), STAT3/5B (Probe 1), STAT3/5B (Probe 2), STAT3/5B (Probe 3), DNMT3A (Probe 1), DNMT3A (Probe 2), and FAS.

CTCLs are cancers of the T lymphocytes that mainly affect the skin but also involve the blood, lymph nodes, or internal organs. Accordingly, CTCL or CTCL with blood involvement may be detected, using the methods disclosed herein, in a variety of biological samples. In some embodiments, the biological sample comprises a blood sample. In some embodiments, the biological sample comprises a tissue sample. In some embodiments, the tissue sample comprises a skin node or a lymph node.

In some embodiments, the biological sample is enriched for CD3+ lymphocytes, CD4+ lymphocytes, and/or CD3+CD4+ lymphocytes. In some embodiments, the biological sample is enriched for CD7− lymphocytes and/or CD26− lymphocytes. It should be understood that CTCL with blood involvement encompasses clinically heterogeneous malignancies including, but not limited to, L-CTCL, Sézary syndrome (SS), mycosis fungoides (MF), follicular mucinosis, mycosis fungoides (MF), patch/plaque mycosis fungoides, folliculotropic mycosis fungoids (F-MF), or tumor-stage mycosis fungoides.

A genetic abnormality associated with CTCL or CTCL with blood involvement may be detected in nucleic acid, for example, DNA and RNA. In some embodiments, the nucleic acid is analyzed using a fluorescence in situ hybridization (FISH) assay. In some embodiments, the nucleic acid is analyzed using a nucleic acid sequencing assay. In some embodiments, the nucleic acid is analyzed using a single nucleotide polymorphism (SNP) array or a transcript array.

Thus, described here is a method of detecting a genetic abnormality in cells obtained from an individual, such as a human, comprising analyzing cells obtained from an individual for the presence or absence of a genetic abnormality in TP53, MYC, RB1, CDKN2A, ATM, STAT3/5B, ARID1A, ZEB1, FAS, CARD11, and DNMT3A, wherein the genetic abnormality comprises at least one deletion in at least one of TP53, RB1, CDKN2A, ATM, ARID1A, ZEB1, FAS, and DNMT3A; at least one amplification in at least one of MYC, STAT3/5B, and CARD11; or at least one deletion in at least one of TP53, RB1, CDKN2A, ATM, ARID1A, ZEB1, FAS, and DNMT3A and at least one amplification in at least one of MYC, STAT3/5B, and CARD11. If at least one deletion is detected in at least one of TP53, RB1, CDKN2A, ATM, ARID1A, ZEB1, FAS, and DNMT3A or at least one amplification is detected in at least one of MYC, STAT3/5B, and CARD11, a genetic abnormality is detected.

In one embodiment, the method is a method of detecting a genetic abnormality associated with cutaneous T cell lymphoma (CTCL) or CTCL with blood involvement, comprising: (a) analyzing a biological sample for a genetic abnormality in TP53, MYC, RB1, CDKN2A, ATM, STAT3/5B, ARID1A, ZEB1, FAS, CARD11, and DNMT3A and (b) determining the presence or absence of a genetic abnormality in TP53, MYC, RB1, CDKN2A, ATM, STAT3/5B, ARID1A, ZEB1, FAS, CARD11, and DNMT3A. If there is a genetic abnormality in at least one of TP53, MYC, RB1, CDKN2A, ATM, STAT3/5B, ARID1A, ZEB1, FAS, CARD11, and DNMT3A, a genetic abnormality associated with CTCL or CTCL with blood involvement is detected. In a specific embodiment, the genetic abnormality comprises at least one deletion in at least one of TP53, RB1, CDKN2A, ATM, ARID1A, ZEB1, FAS, and DNMT3A; at least one amplification in at least one of MYC, STAT3/5B, and CARD11; or at least one deletion in at least one of TP53, RB1, CDKN2A, ATM, ARID1A, ZEB1, FAS, and DNMT3A and at least one amplification in at least one of MYC, STAT3/5B, and CARD11.

In specific embodiments, the genetic abnormality (deletion or amplification) is detected by combining the biological sample with at least one probe selected from ARID1A (Probe 1); ARID1A (Probe 2); ARID1A (Probe 3); CARD11 (Probe 1); CARD11 (Probe 2); CARD11 (Probe 3); ZEB1 (Probe 1); ZEB1 (Probe 2); STAT3/5B (Probe 1); STAT3/5B (Probe 2); STAT3/5B (Probe 3); DNMT3A (Probe 1); DNMT3A (Probe 2); FAS; TP53 Probe; p53/ATM Probe Combination; cMYC Breakapart Probe; RB1 Deletion Probe; and p16 Deletion Probe. Also described herein is a method of determining if an individual has cutaneous T cell lymphoma (CTCL) or CTCL with blood involvement, comprising: (a) analyzing a biological sample obtained from the individual for a genetic abnormality in at least one of TP53, MYC, RB1, CDKN2A, ATM, STAT3/5B, ARID1A, ZEB1, FAS, CARD11, and DNMT3A; (b) assessing (enumerating) the copy number of at least one of the 11 genes (TP53, MYC, RB1, CDKN2A, ATM, STAT3/5B, ARID1A, ZEB1, FAS, CARD11, and DNMT3A) in the biological sample; (c) determining, for each gene for which copy number was assessed, the portion or percent of cells in the sample that show an abnormal gene copy number; and (d) comparing the portion or percent to a reference value or cutoff, wherein if the portion or percent of cells that show an abnormal gene copy number is greater than the reference value or cutoff, the individual has cutaneous T cell lymphoma (CTCL) or CTCL with blood involvement. The genetic abnormality comprises at least one deletion in at least one of TP53, RB1, CDKN2A, ATM, ARID1A, ZEB1, FAS, and DNMT3A; at least one amplification in at least one of MYC, STAT3/5B, and CARD11; or at least one deletion in at least one of TP53, RB1, CDKN2A, ATM, ARID1A, ZEB1, FAS, and DNMT3A and at least one amplification in at least one of MYC, STAT3/5B, and CARD11. If more than about 12% of the population of cells shows an abnormal copy number, the individual from whom the biological sample was obtained is diagnosed as having CTCL or CTCL with blood involvement.

Also described is a method of detecting, in an individual, cutaneous T cell lymphoma (CTCL) or CTCL with blood involvement, comprising: (a) analyzing nucleic acids from a biological sample obtained from an individual for a genetic abnormality in TP53, RB1, CDKN2A, ATM, ARID1A, ZEB1, FAS, and DNMT3A that is a deletion; and for a genetic abnormality in MYC, STAT3/5B, CARD11 that is an amplification, (b) identifying the biological sample having one or more genetic abnormalities in TP53, MYC, RB1, CDKN2A, ATM, STAT3/5B, ARID1A, ZEB1, FAS, CARD11, and DNMT3A as a biological sample having CTCL or CTCL with blood involvement, wherein the genetic abnormality comprises at least one deletion in at least one of TP53, RB1, CDKN2A, ATM, ARID1A, ZEB1, FAS, and DNMT3A; at least one amplification in at least one of MYC, STAT3/5B, and CARD11; or at least one deletion in at least one of TP53, RB1, CDKN2A, ATM, ARID1A, ZEB1, FAS, and DNMT3A and at least one amplification in at least one of MYC, STAT3/5B, and CARD11.

In another embodiment, the method is a method of detecting a genetic abnormality in a biological sample obtained from a human, comprising analyzing nucleic acids (DNA, RNA) from a biological sample for a genetic abnormality that is at least one deletion in TP53, at least one deletion in ARID1A, at least one amplification in MYC and at least one amplification in STAT3/5B, and identifying a biological sample having one or more genetic abnormalities in TP53, MYC, STAT3/5B, and ARID1A as a biological sample from a human having CTCL with blood involvement,

In another embodiment, the method comprises detecting, in a biological sample, a genetic abnormality in TP53 that is a (at least one) deletion; a genetic abnormality in ARID1A that is a (at least one) deletion; a genetic abnormality in MYC that is an (at least one) amplification; and a genetic abnormality in STAT3/5B that is an (at least one) amplification.

In a specific embodiment, the biological sample is a blood sample or a tissue sample that comprises a skin node or a lymph node. The biological sample, in some embodiments, is enriched for CD3+ lymphocytes, CD4+ lymphocytes, and/or CD3+CD4+ lymphocytes. The biological sample, in some embodiments, is further enriched for CD7− lymphocytes and/or CD26− lymphocytes.

In some embodiments, CTCL with blood involvement is leukemic cutaneous T cell lymphoma (L-CTCL), Sézary syndrome (SS), mycosis fungoides (MF), or follicular mucinosis. The mycosis fungoides (MF) is patch/plaque mycosis fungoides, folliculotropic mycosis fungoids (F-MF), or tumor-stage mycosis fungoides. In any of the embodiments, the nucleic acid can be analyzed using a fluorescence in situ hybridization (FISH) assay. Alternatively, assessment of nucleic acids can be done by nucleic acid sequencing or through use of a single nucleotide polymorphism (SNP) array or a transcript array.

One embodiment described is a method of detecting a genetic abnormality associated with cutaneous T cell lymphoma (CTCL) or CTCL with blood involvement, comprising:

(a) analyzing a biological sample for a genetic abnormality in TP53, MYC, RB1, CDKN2A, ATM, STAT3/5B, ARID1A, ZEB1, FAS, CARD11, and DNMT3A; and

(b) determining the presence or absence of a genetic abnormality in TP53, MYC, RB1, CDKN2A, ATM, STAT3/5B, ARID1A, ZEB1, FAS, CARD11, and DNMT3A, wherein if there is a genetic abnormality in at least one of TP53, MYC, RB1, CDKN2A, ATM, STAT3/5B, ARID1A, ZEB1, FAS, CARD11, and DNMT3A, a genetic abnormality associated with CTCL with blood involvement is detected.

A further embodiment is a method of detecting, in a biological sample comprising cells obtained from an individual, a genetic abnormality (a deletion or an amplification) associated with cutaneous T cell lymphoma (CTCL) or CTCL with blood involvement, comprising assessing TP53, MYC, RB1, CDKN2A, ATM, STAT3/5B, ARID1A, ZEB1, FAS, CARD11, and DNMT3A for a genetic abnormality, wherein if a genetic abnormality is present in any of/in at least one of TP53, MYC, RB1, CDKN2A, ATM, STAT3/5B, ARID1A, ZEB1, FAS, CARD11, and DNMT3A, cells in the biological sample comprise a genetic abnormality associated with CTCL or CTCL with blood involvement. In some embodiments, the deletion or abnormality is detected by combining the biological sample with at least one probe selected from ARID1A (Probe 1); ARID1A (Probe 2); ARID1A (Probe 3); CARD11 (Probe 1); CARD11 (Probe 2); CARD11 (Probe 3); ZEB1 (Probe 1); ZEB1 (Probe 2); STAT3/5B (Probe 1); STAT3/5B (Probe 2); STAT3/5B (Probe 3); DNMT3A (Probe 1); DNMT3A (Probe 2); FAS; TP53 Probe; p53/ATM Probe Combination; cMYC Breakapart Probe; RB1 Deletion Probe; and p16 Deletion Probe.

Another embodiment is a method of determining if an individual (e.g., a human) has cutaneous T cell lymphoma (CTCL) by analyzing a biological sample obtained from the individual for a genetic abnormality in at least one of TP53, MYC, RB1, CDKN2A, ATM, STAT3/5B, ARID1A, ZEB1, FAS, CARD11, and DNMT3A; assessing (enumerating) the copy number of at least one of the 11 genes (TP53, MYC, RB1, CDKN2A, ATM, STAT3/5B, ARID1A, ZEB1, FAS, CARD11, and DNMT3A) in the biological sample; determining, for each gene for which copy number was assessed, the portion or percent of cells that show an abnormal gene copy number; and comparing the portion or percent to a reference value or cutoff. If the portion or percent of cells that show an abnormal gene copy number is greater than the reference value or cutoff, the individual has cutaneous T cell lymphoma (CTCL). In this embodiment, the genetic abnormality comprises at least one deletion in at least one of TP53, RB1, CDKN2A, ATM, ARID1A, ZEB1, FAS, and DNMT3A; at least one amplification in at least one of MYC, STAT3/5B, and CARD11; or at least one deletion in at least one of TP53, RB1, CDKN2A, ATM, ARID1A, ZEB1, FAS, and DNMT3A and at least one amplification in at least one of MYC, STAT3/5B, and CARD11. If more than about 12% of the population of cells shows an abnormal copy number, the individual from whom the biological sample was obtained is diagnosed as having CTCL or CTCL with blood involvement. The deletion or abnormality is detected, for example, by combining the biological sample with at least one probe selected from ARID1A (Probe 1); ARID1A (Probe 2); ARID1A (Probe 3); CARD11 (Probe 1); CARD11 (Probe 2); CARD11 (Probe 3); ZEB1 (Probe 1); ZEB1 (Probe 2); STAT3/5B (Probe 1); STAT3/5B (Probe 2); STAT3/5B (Probe 3); DNMT3A (Probe 1); DNMT3A (Probe 2); FAS; TP53 Probe; p53/ATM Probe Combination; cMYC Breakapart Probe; RB1 Deletion Probe; and p16 Deletion Probe.

A further approach to detecting cutaneous T cell lymphoma (CTCL) is a method comprising: (a) analyzing nucleic acids from a biological sample for a genetic abnormality in TP53, MYC, RB1, CDKN2A, ATM, STAT3/5B, ARID1A, ZEB1, FAS, CARD11, and DNMT3A; and (b) identifying the biological sample having one or more genetic abnormalities in TP53, MYC, RB1, CDKN2A, ATM, STAT3/5B, ARID1A, ZEB1, FAS, CARD11, and DNMT3A as a biological sample having CTCL, wherein the genetic abnormality comprises at least one deletion in at least one of TP53, RB1, CDKN2A, ATM, ARID1A, ZEB1, FAS, and DNMT3A; at least one amplification in at least one of MYC, STAT3/5B, and CARD11; or at least one deletion in at least one of TP53, RB1, CDKN2A, ATM, ARID1A, ZEB1, FAS, and DNMT3A and at least one amplification in at least one of MYC, STAT3/5B, and CARD11. The deletion or abnormality is detected, for example, by combining the biological sample with at least one probe selected from ARID1A (Probe 1); ARID1A (Probe 2); ARID1A (Probe 3); CARD11 (Probe 1); CARD11 (Probe 2); CARD11 (Probe 3); ZEB1 (Probe 1); ZEB1 (Probe 2); STAT3/5B (Probe 1); STAT3/5B (Probe 2); STAT3/5B (Probe 3); DNMT3A (Probe 1); DNMT3A (Probe 2); FAS; TP53 Probe; p53/ATM Probe Combination; cMYC Breakapart Probe; RB1 Deletion Probe; and p16 Deletion Probe.

A further embodiment of the method of detecting cutaneous T cell lymphoma (CTCL), comprises (a) analyzing nucleic acids (DNA, RNA) from a biological sample for a genetic abnormality in TP53, MYC, STAT3/5B, and ARID1A; and (b) identifying a biological sample having one or more genetic abnormalities in TP53, MYC, STAT3/5B, and ARID1A as a biological sample having CTCL with blood involvement, wherein the genetic abnormality comprises at least one deletion in TP53, at least one deletion in ARID1A, at least one amplification in MYC or at least one amplification in STAT3/5B. In any of the embodiments, the biological sample comprises a blood sample or a tissue sample, such as a tissue sample that comprises a skin node or a lymph node. The deletion or abnormality is detected, for example, by combining the biological sample with at least one probe selected from ARID1A (Probe 1); ARID1A (Probe 2); ARID1A (Probe 3); STAT3/5B (Probe 1); STAT3/5B (Probe 2); STAT3/5B (Probe 3); TP53 Probe; cMYC Breakapart Probe. The biological sample is enriched for CD3+ lymphocytes, CD4+ lymphocytes, and/or CD3+CD4+ lymphocytes and, in some embodiments, further enriched for CD7− lymphocytes and/or CD26− lymphocytes. CTCL with blood involvement is, for example, leukemic cutaneous T cell lymphoma (L-CTCL), Sézary syndrome (SS), mycosis fungoides (MF, e.g., is patch/plaque mycosis fungoides, folliculotropic mycosis fungoids (F-MF), or tumor-stage mycosis fungoide), or follicular mucinosis. Nucleic acid can be analyzed using a fluorescence in situ hybridization (FISH) assay, nucleic acid sequencing assay, single nucleotide polymorphism (SNP) array or a transcript array.

### BRIEF DESCRIPTION OF THE DRAWINGS

The patent or application file contains at least one drawing executed in color. Copies of this patent or patent application publication with color drawings will be provided by the Patent Office upon request and payment of the necessary fee.

FIG. 1. L-CTCL FISH panel composition and probe microscopy. Panel A: Table of genes, chromosomal locations, and fluorescent colors for 11 clinically validated probes used in the CTCL FISH panel. Panels B-G: Post-hybridization microscopy images using 11-probe FISH panel on peripheral blood samples from patients with L-CTCL and abnormal FISH results. Panel B: Field with cell showing normal copy number of two TP53 (red) signals and two ATM (green) signals, and two cells with deletion of one copy of TP53. Panel C: Cells with two copies of CDKN2A (red) and 9q12 control probe (green). Panel D: Two cells with two copies of RB1 (red) and 13q control (green). Panel E: Signals for DMNT3A (green), CARD11 (aqua), and FAS (red), both cells showing two copies of each. Panel F: Cell on top with two copies of MYC stained with the red and green probes (both for MYC); cell below showing four copies of MYC. Panel G: Signals for ARID1A (red), ZEB1 (green), and STAT3/5B (aqua); one cell with normal copy numbers and two cells with homozygous deletion of ZEB1 and amplification of STAT3/5B. Black bar=10 μm.

FIG. 2. Distribution of deletions and amplifications detected by FISH. Panel A: Heatmap of deletions and amplification in CTCL driver genes detected by the 11-probe CTCL FISH panel among 10 patients with SS, 1 patient with F-MF, 1 patient with MF-T, and 2 patients with MF. Each column shows FISH status for one patient. Panel B: Comparison of proportion+/− SEM of SS patients with abnormal copy numbers in each gene by FISH (n=10) versus CTCL exome sequencing (n=40) on non-overlapping patient cohorts. No significant differences were found for any gene (Fisher's exact test, p>0.1). *For STAT3/5B FISH probe, comparator gene from exome data is STAT5B.

FIG. 3. Distribution of deletions and amplifications detected by FISH, with diagnostic subset indicated. Panel A: Heatmap of deletions and amplification in CTCL driver genes detected by the 11-probe CTCL FISH panel among 10 patients with SS, 1 patient with F-MF, 1 patient with MF-T, and 2 patients with MF. Each column shows FISH status for one patient. Diagnostic subset of four genes (TP53, ARID1A, MYC, STAT3/5B) is indicated. Panel B: Comparison of proportion+/− SEM of SS patients with abnormal copy numbers in each gene by FISH (n=10) versus CTCL exome sequencing (n=40) on non-overlapping patient cohorts. No significant differences were found for any gene (Fisher's exact test, p>0.1). *For STAT3/5B FISH probe, comparator gene from exome data is STAT5B.

FIG. 4 shows results of sample sorting using FACS. Flow cytometric graphs show that the biological sample may be enriched for CD3+ lymphocytes, CD4+ lymphocytes, CD3+CD4+ lymphocytes, CD3+CD4+CD7− lymphocytes and/or CD3+CD4+CD26-lymphocytes.

### DETAILED DESCRIPTION

Currently, no clinical tests are widely used to assess mutational burden in cutaneous T-cell lymphoma (CTCL) or CTCL with leukemic (L-CTCL) involvement, such as in Sézary syndrome (SS), and routine blood analysis offers limited guidance for assessing prognosis and optimizing treatment within the highly variable population of affected patients. In addition, CTCL cells involve the skin and may develop into various types of skin lesions (such as patches, plaques and tumors), may involve the lymph nodes, and may involve various visceral organs. Aside from the demonstration of T cell clonality by examination of the T cell receptor (TCR) status by Southern hybridization, polymerase chain reaction (PCR), or DNA/RNA sequencing (e.g., by high throughput sequencing (HTS)), there are limited options for molecular testing for CTCL involvement in the blood, skin, lymph node, or visceral organs.

As described herein, certain biomarkers (e.g., genetic abnormalities) are differentially present in patients with cutaneous T cell lymphoma (CTCL) or CTCL with leukemic (L-CTCL) involvement than in individuals who do not have CTCL (control individuals). For example, as described further herein, TP53, MYC, STAT3/5B, and ARID1A are biomarkers indicative of CTCL or L-CTCL. It was determined that samples from patients having CTCL or L-CTCL showed at least one deletion in TP53 and/or ARID1A or at least one amplification in MYC and/or STAT3/5B. Accordingly, genetic abnormalities in such biomarkers (e.g., TP53, MYC, STAT3/5B, and ARID1A) correlated with presence of CTCL or L-CTCL in samples from cancer patients.

Some aspects of the disclosure provide methods for analyzing samples from individuals suspected of having CTCL based on the presence or absence of a genetic abnormality in one or more biomarkers (e.g., TP53, ATM, CDKN2A, RB1, MYC, FAS, DMNT3A, CARD11, ARID1A, ZEB1, and STAT3/5B). Such assay methods may be used for clinical purposes, e.g., for determining presence of CTCL or L-CTCL in a sample obtained from an individual, identifying individuals having CTCL or L-CTCL, selecting a candidate for treatment, monitoring CTCL or L-CTCL progression, assessing the efficacy of a treatment against CTCL or L-CTCL, determining a course of treatment, and assessing whether an individual is at risk for relapse of CTCL or L-CTCL. The assay methods described herein may also be useful for non-clinical applications, for example, for research purposes, including, e.g., studying the mechanism of CTCL or L-CTCL development and/or biological pathways/processes involved in CTCL or L-CTCL, and developing new therapies for CTCL or L-CTCL based on such studies.

### Biomarkers

The methods described herein are based, at least in part, on biomarkers (e.g., genetic abnormalities) that are differentially present in patients that had cutaneous T cell lymphoma (CTCL) or CTCL with leukemic (L-CTCL) involvement, compared to control patients.

As used herein, the term “biomarker” or “biomarker set” indicative of CTCL or L-CTCL refers to a genetic abnormality (e.g., a gene copy number alteration). For example, a biomarker that is indicative of CTCL or L-CTCL has a gene copy number alteration (e.g., amplification or deletion) in a sample from an individual (e.g., a sample from an individual that has or is at risk for CTCL or L-CTCL) relative to the gene copy number of the same biomarker in a control sample (e.g., a sample from an individual who does not have or is not at risk for CTCL or L-CTCL).

Examples of biomarkers indicative of CTCL or L-CTCL are provided in Table 1. The biomarkers described herein may have a gene copy number in a sample obtained from an individual (e.g., a patient) that has CTCL or L-CTCL that deviates (e.g., amplified or deleted) when compared to the gene copy number of the same biomarker in a sample from a normal individual (e.g., an individual who does not have or is not at risk for CTCL or L-CTCL) by at least 10% (e.g., 20%, 30%, 50%, 80%, 100%, 2-fold, 5-fold, 10-fold, 20-fold, 50-fold, 100-fold or more). Such a biomarker or set of biomarkers may be used in both diagnostic/prognostic applications and non-clinical applications (e.g., for research purposes).

TABLE 1
Exemplary biomarkers.

| Gene | Chromosomal location |
|---|---|
| TP53 | 17p13.1 |
| ATM | 11q22.3 |
| CDKN2A | 9p21.3 |
| RB1 | 13q14.2 |
| MYC | 8q24.21/8q24.21 |
| FAS | 10q24.1 |
| DMNT3A | 2p23 |
| CARD11 | 7p22 |
| ARID1A | 1p35.3 |
| ZEB1 | 10p11.2 |
| STAT3/5B | 17q21.31/17q11.2 |

In some embodiments, methods described herein provide determining a genetic abnormality (e.g., altered gene copy number, such as amplification or deletion) in from 1 to 11 biomarkers indicative of CTCL or L-CTCL. In some embodiments, methods described herein provide determining a genetic abnormality in between 2 to 11 biomarkers, between 3 to 11 biomarkers, between 4 to 11 biomarkers, between 5 to 11 biomarkers, between 6 to 11 biomarkers, between 7 to 11 biomarkers, between 8 to 11 biomarkers, between 9 to 11, or between 10 to 11 biomarkers indicative of CTCL or L-CTCL. In some embodiments, methods described herein provide determining a genetic abnormality in between 1 to 10 biomarkers, between 1 to 9 biomarkers, between 1 to 8 biomarkers, between 1 to 7 biomarkers, between 1 to 6 biomarkers, between 1 to 5 biomarkers, between 1 to 4 biomarkers, between 1 to 3, or between 1 to 2 biomarkers indicative of CTCL or L-CTCL.

In some embodiments, methods described herein provide determining a genetic abnormality in at least one biomarker, at least 2 biomarkers, at least 3 biomarkers, at least 4 biomarkers, at least 5 biomarkers, at least 6 biomarkers, at least 7 biomarkers, at least 8 biomarkers, at least 9 biomarkers, at least 10 biomarkers, or at least 11 biomarkers indicative of CTCL or L-CTCL.

In some embodiments, methods described herein provide determining a genetic abnormality in at least one biomarker selected from a group of biomarkers indicative of CTCL or L-CTCL. In some embodiments, the method determines a genetic abnormality in one (at least one) of the following biomarkers: TP53, MYC, RB1, CDKN2A, ATM, STAT3/5B, ARID1A, ZEB1, FAS, CARD11, and DNMT3A. In some embodiments, the method determines a genetic abnormality in one (at least one) of the following biomarkers: TP53, MYC, STAT3/5B, and ARID1A.

Any number and/or combination of the biomarkers listed herein or the groups of biomarkers listed herein may be used in the described methods. For example, 1, 2, 3, 4, 5, 6, 7, 8, 9, 10, or 11 or more of the biomarkers listed herein can be used in the method.

### Utilities of Biomarkers

Any of the biomarkers described herein, either taken alone or in combination (e.g., at least two biomarkers, at least three biomarkers, or more biomarkers), can be used in the methods also described herein for analyzing a sample from an individual that has or is at risk for CTCL or L-CTCL. Results obtained from such methods can be used in either clinical applications or non-clinical applications, including, but not limited to, those described herein.

(i) Analysis of Biological Samples

Any sample that may contain a biomarker (e.g., a biological sample such as blood) can be analyzed by the methods described herein. The methods described herein may include providing a sample obtained from an individual. In some embodiments, the sample may be from an in vitro assay, for example, an in vitro cell culture (e.g., an in vitro culture of T cells).

As used herein, a “sample” refers to a composition that comprises biological materials such as (but not limited to) tissue, blood, plasma, nucleic acid, or protein from an individual. A sample can be an initial unprocessed sample taken from a subject as well as subsequently processed, e.g., partially purified or preserved forms. Samples include tissue, organ, skin, lymph node, blood, plasma, tears, mucus, or bone marrow. In some embodiments, a tissue sample comprises a skin node or a lymph node.

In some embodiments, a sample comprises leukocytes. In some embodiments, the lymphocytes may be sorted to isolate T cell populations consistent with CTCL or CTCL with blood involvement. In some embodiments, T cell populations are enriched for CD3+ lymphocytes. In some embodiments, T cell populations are enriched for CD4+ lymphocytes. In some embodiments, T cell populations are enriched for CD3+CD4+ lymphocytes. In some embodiments, T cell populations are enriched for CD7− lymphocytes. In some embodiments, T cell populations are enriched for CD26− lymphocytes. In some embodiments, T cell populations are enriched for CD7-CD26− lymphocytes.

In some embodiments, a sample comprises T cells. In some embodiments, the T cells are from the blood. In some embodiments, the T cells are from skin, lymph nodes, or visceral organs.

In some embodiments, multiple (e.g., at least 2, 3, 4, 5, or more) samples may be collected from subject, over time or at particular time intervals, for example to assess the disease progression or evaluate the efficacy of a treatment.

A sample can be obtained from a subject using any means known in the art. In some embodiments, the sample is obtained from the subject by removing the sample (e.g., a blood sample) from the subject. In some embodiments, the sample is obtained from the subject by a surgical procedure. In some embodiments, the sample is obtained from the subject by a biopsy. In some embodiments, the sample is obtained from the subject by aspirating, brushing, scraping, or a combination thereof. In some embodiments, the sample is obtained from a human.

The term “subject” refers to a subject in need of the analysis described herein. In some embodiments, the subject is a patient. In some embodiments, the subject is a human. In some embodiments, a subject is at risk for CTCL or L-CTCL (whether known or unknown). Such a subject may exhibit one or more risk factors associated with CTCL or L-CTCL. Exemplary risk factors include, but are not limited to, age, gender, ethnicity, exposure to certain chemicals and drugs, radiation exposure, a weakened immune system or autoimmune disease, obesity, and having certain infections.

Alternatively, the subject in need of the analysis described herein may be a patient who has or is at risk for CTCL or L-CTCL (known or unknown). Such a subject may currently have CTCL or L-CTCL, or may have had CTCL or L-CTCL in the past. Such a subject may be at risk for CTCL or L-CTCL. In some examples, the subject is a human patient who is being treated for CTCL or L-CTCL with, for example, chemotherapy, corticosteroid, radiation, phototherapy, or retinoids. In other instances, such a human patient may be free of such a treatment (i.e., is not being treated currently).

Examples of CTCL include, without limitation, CTCL with blood involvement (L-CTCL), Sézary syndrome (SS), mycosis fungoides (MF), follicular mucinosis, mycosis fungoides (MF), patch/plaque mycosis fungoides, folliculotropic mycosis fungoids (F-MF), or tumor-stage mycosis fungoides. In some embodiments, CTCL comprises CTCL involving the skin, lymph nodes, or the visceral organs.

Any of the samples described herein can be subject to analysis using the assay methods described herein, which involve determining the presence or absence of a genetic abnormality in one or more biomarkers as described herein. Genetic abnormalities (e.g., a copy number alteration) in a biomarker disclosed herein can be assessed using conventional assays or those described herein.

As used herein, the terms “determining” or “measuring,” or alternatively “detecting,” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject.

Assays for determining the presence or absence of a genetic abnormality in a sample (e.g., abnormal copy number) include, but are not limited to, fluorescence in situ hybridization (FISH), reverse transcriptase polymerase chain reaction (RT-PCR), comparative genomic hybridization, array comparative genomic hybridization (aCGH), SNP array technologies and immunohistochemistry (IHC). In some embodiments, determining the presence or absence of a genetic abnormality comprises measuring mRNA. In some embodiments, the expression level of mRNA encoding a biomarker can be measured using polymerase chain reaction (PCR), reverse transcriptase-PCR (RT-PCR), in situ PCR, quantitative PCR (Q-PCR), real-time quantitative PCR (RT Q-PCR), in situ hybridization, Southern blot, Northern blot, sequence analysis, microarray analysis, detection of a reporter gene, or other DNA/RNA hybridization platforms.

Any binding agent that specifically binds to a desired biomarker may be used in the methods and kits described herein to measure a genetic abnormality (e.g., altered gene copy number, such as amplification or deletion) in a sample. In some embodiments, the binding agent is a probe that specifically binds to a desired biomarker. In some embodiments, the probe may be one or more oligonucleotides complementary to a coding nucleic acid or a portion thereof. In some embodiments, the probe is commercially available. In some embodiments, the probe is provided herein. In some embodiments, a sample may be contacted, simultaneously or sequentially, with more than one probe that binds different biomarkers (e.g., multiplexed analysis). Examples of probes provided herein and used in the analysis described herein are provided in Table 2 and Table 3.

TABLE 2
Probes provided herein.

| | | GENCODE | GENCODE | |
|---|---|---|---|---|
| | Chromosome | Sequence | Sequence | Probe |
| Probe | locus | Start | End | size |
| ARID1A (Probe 1) | 1p36.11 | 26881401 | 27008254 | 126854 |
| ARID1A (Probe 2) | 1p36.11 | 26982949 | 27158028 | 175080 |
| ARID1A (Probe 3) | 1p36.11 | 27148799 | 27322151 | 173353 |
| CARD11 (Probe 1) | 7p22.3-p22.2 | 2764802 | 2905045 | 140244 |
| CARD11 (Probe 2) | 7p22.2 | 2874600 | 3104101 | 229502 |
| CARD11 (Probe 3) | 7p22.2 | 3069406 | 3216536 | 147131 |
| ZEB1 (Probe 1) | 10p11.22 | 31535329 | 31720845 | 185517 |
| ZEB1 (Probe 2) | 10p11.22 | 31689724 | 31901675 | 211952 |
| STAT3/5B | 17q21.2 | 40189722 | 40313325 | 123604 |
| (Probe 1) | | | | |
| STAT3/5B | 17q21.2 | 40319736 | 40515329 | 195594 |
| (Probe 2) | | | | |
| STAT3/5B | 17q21.2 | 40510068 | 40672021 | 161954 |
| (Probe 3) | | | | |
| DNMT3A (Probe 1) | 2p23.3 | 25352042 | 25478141 | 126100 |
| DNMT3A (Probe 2) | 2p23.3 | 25421261 | 25595905 | 174645 |
| FAS | 10q23.31 | 90665836 | 90864834 | 198999 |

To determine a genetic abnormality in a target biomarker, a sample can be in contact with a binding agent under suitable conditions. In general, the term “contact” refers to an exposure of the binding agent with the sample or cells collected therefrom for suitable period sufficient for the formation of complexes between the binding agent and the target biomarker in the sample, if any. In some embodiments, the contacting is performed by capillary action in which a sample is moved across a surface of the support membrane.

In some embodiments, the assays may be performed on low-throughput platforms, including single assay format. For example, a low throughput platform may be used to measure the presence or absence of a genetic abnormality in a sample (e.g., tissue and/or blood) for diagnostic methods, monitoring of disease and/or treatment progression, and/or predicting whether a disease or disorder may benefit from a particular treatment.

In some embodiments, it may be necessary to immobilize a binding agent to the support member. Methods for immobilizing a binding agent will depend on factors such as the nature of the binding agent and the material of the support member and may require particular buffers. Such methods will be evident to one of ordinary skill in the art. For example, the biomarker set in a sample as described herein may be measured using any of the kits and/or detecting devices which are also described herein.

The type of detection assay used for the detection and/or quantification of a biomarker such as those provided herein may depend on the particular situation in which the assay is to be used (e.g., clinical or research applications), on the kind and number of biomarkers to be detected, and/or on the kind and number of patient samples to be run in parallel, to name a few parameters.

The methods described herein may be used for both clinical and non-clinical purposes. Some examples are provided herein.

(ii) Diagnostic and/or Prognostic Applications

The levels of one or more of the biomarkers in a sample derived from a subject, measured by the assay methods described herein, can be used for various clinical purposes, for example, detecting cutaneous T-cell lymphoma (CTCL) or CTCL with leukemic (L-CTCL) involvement, identifying a subject having CTCL or L-CTCL, monitoring the progress of CTCL or L-CTCL in a subject, assessing the efficacy of a treatment for CTCL or L-CTCL in a subject, identifying patients suitable for a particular treatment, and/or predicting disease relapse in a subject. Accordingly, described herein are diagnostic and prognostic methods for CTCL or L-CTCL based on the presence or absence of a genetic abnormality in one or more biomarkers described herein, e.g., TP53, MYC, RB1, CDKN2A, ATM, STAT3/5B, ARID1A, ZEB1, FAS, CARD11, and DNMT3A.

When needed, the presence or absence of a genetic abnormality in a biomarker in a sample as determined by methods described herein may be normalized with an internal control in the same sample or with a standard sample (having a predetermined amount of the biomarker) to obtain a normalized value. Either the raw value or the normalized value of the biomarker can then be compared with that in a reference sample or a control sample. A deviated value of the biomarker (e.g., amplification or deletion) in a sample obtained from a subject as relative to the value of the same biomarker in the reference or control sample is indicative of presence of CTCL or L-CTCL in the sample. A subject carrying one or more genetic abnormalities in one or more biomarkers indicates that the subject may have CTCL or L-CTCL.

In some embodiments, the presence or absence of a genetic abnormality (e.g., gene copy number) in the biomarker in a sample obtained from a subject can be compared to a predetermined threshold for that biomarker, a deviation from which may indicate that the subject is carrying a genetic abnormality, and thus may have CTCL or L-CTCL.

The control sample or reference sample may be a sample obtained from a healthy individual. Alternatively, the control sample or reference sample contains a known genetic abnormality in the biomarker to be assessed. In some embodiments, the control sample or reference samples is a biological sample obtained from a control subject.

As used herein, a control subject may be a healthy individual, e.g., an individual that is apparently free of CTCL or L-CTCL at the time the presence or absence of a genetic abnormality (e.g., gene copy number) is measured or has no history of the disease. A control subject may also represent a population of healthy subjects, who preferably would have features (e.g., age, gender, ethnic group) matched with those of the subject being analyzed by a method described herein. In some embodiments, a control subject has CTCL without leukemic involvement. In some embodiments, a control subject has a lymphoma without blood involvement.

The control value can be a predetermined amount or threshold. Such a predetermined value can represent the presence or absence of a genetic abnormality in a biomarker in a population of subjects that do not have or are not at risk for CTCL or L-CTCL (e.g., the presence or absence of a genetic abnormality in the population of healthy subjects). It can also represent the presence or absence of a genetic abnormality in a biomarker in a population of subjects that have CTCL or L-CTCL.

The predetermined value can take a variety of forms. For example, it can be a single cutoff value, such as a median or mean. In some embodiments, such a predetermined value can be established based upon comparative groups, such as where one defined group is known to have CTCL or L-CTCL and another defined group is known to not have CTCL or L-CTCL. Alternatively, the predetermined value can be a range, for example, a range representing the copy number of the biomarker in a control population.

The control value as described herein can be determined by routine technology. In some examples, the control value can be obtained by performing a conventional method (e.g., the same assay for obtaining the level of the protein a test sample as described herein) on a control sample as also described herein. In other examples, a gene copy number can be obtained from members of a control population and the results can be analyzed by, e.g., a computational program, to obtain the control value (a predetermined value) that represents the copy number of the biomarker in the control population.

By comparing the gene copy number of a biomarker in a sample obtained from a candidate subject to the reference value as described herein, it can be determined as to whether the candidate subject has or is at risk for CTCL or L-CTCL.

In some embodiments, if the gene copy number of biomarker(s) in a sample of the candidate subject is abnormal as compared to the reference value, the candidate subject might be identified as having or at risk for CTCL or L-CTCL. In some embodiments, if the gene copy number of biomarker(s) in a sample of the candidate subject increased (e.g., gene amplification) as compared to the reference value, the candidate subject might be identified as having or at risk for CTCL or L-CTCL. In some embodiments, if the gene copy number of biomarker(s) in a sample of the candidate subject decreased (e.g., gene deletion) as compared to the reference value, the candidate subject might be identified as having or at risk for CTCL or L-CTCL. When the reference value represents the value range of the copy number of the biomarker in a population of subjects that carry a genetic abnormality and/or have CTCL or L-CTCL, the value of biomarker in a sample of a candidate falling in the range indicates that the candidate subject has or is at risk for CTCL or L-CTCL.

As used herein, “an abnormal gene copy number” or “a gene copy number that is increased” or “a gene copy number that is decreased” means that the gene copy number of the biomarker is higher (e.g., amplification) or lower (e.g., deletion) than a reference value, such as a predetermined threshold of a gene copy number of the biomarker in a control sample. Controls are described in detail herein.

An abnormal gene copy number of a biomarker includes a gene copy number that is for example, 1%, 5%, 10%, 20%, 30%, 40%, 50%, 60%, 70%, 80%, 90%, 100%, 150%, 200%, 300%, 400%, 500% or more above a reference value. In some embodiments, the gene copy number of the biomarker in the test sample is at least 1.1, 1.2, 1.3, 1.4, 15, 1.6, 1.7, 1.8, 1.9, 2, 2.5, 3, 3.5, 4, 4.5, 5, 5, 6, 7, 8, 9, 10, 50, 100, 150, 200, 300, 400, 500, 1000, 10000-fold or more higher than the gene copy of the biomarker in a reference sample.

In some embodiments, a percent or portion of cells comprising an abnormal gene copy number for each biomarker is determined and compared with a reference value. In some embodiments, if the percent of cells having an abnormal gene copy number of biomarker(s) in a sample of the candidate subject is greater than about 12%, the candidate subject might be identified as having or at risk for CTCL or L-CTCL.

In some embodiments, the percent of cells having an abnormal gene copy number of a biomarker is greater than about 3%, greater than about 6%, greater than about 12%, greater than about 24%, greater than about 36%, greater than about 48%, greater than about 60%, greater than about 72%, greater than about 84%, or greater than about 96%.

In some embodiments, if the percent of cells having an abnormal gene copy number of biomarker(s) in a sample of the candidate subject is greater than about 6%, the candidate subject might be identified as having or at risk for CTCL or L-CTCL.

In some embodiments, the candidate subject is a human patient having a symptom of CTCL or L-CTCL. For example, the subject has a scaly red rash or light or dark patches in areas of the body that are not usually exposed to the sun, thin, reddened, eczema-like rash, thickened scaly, red skin (e.g., plaques), psoriasis-like rash, skin ulcers, skin tumors, itching, or a combination thereof. In other embodiments, the subject has no symptom of CTCL or L-CTCL at the time the sample is collected, has no history of a symptom of CTCL or L-CTCL, or no history of CTCL or L-CTCL. In yet other embodiments, the subject is resistant to a chemotherapy, a radiation therapy, immunotherapy, or a combination thereof.

A subject identified in the methods described herein as carrying a genetic abnormality in a biomarker or having CTCL or L-CTCL may be subject to a suitable treatment, such as treatment with a chemotherapy, as described herein.

The assay methods and kits described herein also can be applied for evaluation of the efficacy of a treatment for CTCL or L-CTCL, such as those described herein, given the correlation between the presence or absence of a genetic abnormality in one or more of the biomarkers and such cancers. For example, multiple samples (e.g., blood samples) can be collected from a subject to whom a treatment is performed either before and after the treatment or during the course of the treatment. The presence or absence of a genetic abnormality in one or more of the biomarkers can be measured by any of the assay methods as described herein and values (e.g., gene copy numbers) of a biomarker can be determined accordingly.

For example, if an elevated gene copy number of a biomarker indicates that a subject has CTCL or L-CTCL and the gene copy number of the biomarker decreases after the treatment or over the course of the treatment (the copy number of the biomarker in a later collected sample as compared to that in an earlier collected sample), it indicates that the treatment is effective. In other examples, if an decreased gene copy number of a biomarker indicates that a subject has CTCL or L-CTCL and the gene copy number of the biomarker increase after the treatment or over the course of the treatment (the copy number of the biomarker in a later collected sample as compared to that in an earlier collected sample), it indicates that the treatment is effective.

In some examples, the treatment involves an effective amount of a therapeutic agent, such as a chemotherapeutic agent. Examples of the chemotherapeutic agents include, but are not limited to, mechlorethamine HCl, methotrexate, pralatrexate, gemcitabine, pentostatin, liposomal doxorubicin, chlorambucil, cyclophosphamide, etoposide, temozolomide, alemtuzumab, denileukin diftitox, vorinostat, and romidepsin.

If the subject is identified as not responsive to the treatment, a higher dose and/or frequency of dosage of the therapeutic agent are administered to the subject identified. In some embodiments, the dosage or frequency of dosage of the therapeutic agent is maintained, lowered, or ceased in a subject identified as responsive to the treatment or not in need of further treatment. Alternatively, a different treatment can be applied to the subject who is found as not responsive to the first treatment.

In other embodiments, a genetic abnormality of a biomarker or biomarker set can also be relied on to identify CTCL or L-CTCL that may be treatable, for example by a chemotherapeutic agent. To practice this method, a gene copy number of a biomarker in a sample collected from a subject (e.g., a blood sample) having CTCL or L-CTCL can be measured by a suitable method, e.g., those described herein such as a FISH assay. If the gene copy number of the biomarker is elevated or decreased from the reference value, it indicates that a chemotherapeutic agent may be effective in treating the disease. If the disease is identified as being susceptible (can be treated by) to a chemotherapeutic agent, the method can further comprise administering to the subject having the disease an effective amount of a chemotherapeutic agent, such as mechlorethamine HCl, methotrexate, pralatrexate, gemcitabine, pentostatin, liposomal doxorubicin, chlorambucil, cyclophosphamide, etoposide, temozolomide, alemtuzumab, denileukin diftitox, vorinostat, and romidepsin.

Also within the scope of the present disclosure are methods of evaluating the severity of a CTCL or L-CTCL. For example, as described herein, CTCL or L-CTCL may be in the quiescent state (remission), during which the subject does not experience symptoms of the disease. CTCL or L-CTCL relapses are typically recurrent episodes in which the subject may experience a symptom of L-CTCL including, but not limited to, a scaly red rash or light or dark patches in areas of the body that are not usually exposed to the sun, thin, reddened, eczema-like rash, thickened scaly, red skin (e.g., plaques), psoriasis-like rash, skin ulcers, skin tumors, itching, or a combination thereof. In some embodiments, a gene copy number of one or more biomarkers is indicative of whether the subject will experience, is experiencing, or will soon experience a relapse. In some embodiments, the methods involve comparing the gene copy number of a biomarker in a sample obtained from a subjecting having CTCL or L-CTCL to the level of the biomarker in a sample from the same subject, for example a sample obtained from the same subject at remission or a sample obtained from the same subject during a relapse.

(iii) Non-Clinical Applications

Further, a genetic abnormality in any of the biomarkers described herein may be applied for non-clinical uses including, for example, for research purposes. In some embodiments, the methods described herein may be used to study cell behavior and/or cell mechanisms (e.g., the discovery of novel biological pathways or processes related to disease development, for example, cutaneous T cell lymphoma (CTCL) or cutaneous T cell lymphoma with blood involvement development.

In some embodiments, a genetic abnormality in any of the biomarkers described herein may be relied on in the development of new therapeutics for CTCL or CTCL with blood involvement. For example, a genetic abnormality in a biomarker may be determined in samples obtained from a subject who has been administered a new therapy (e.g., a clinical trial). In some embodiments, a genetic abnormality in any of the biomarkers may indicate the efficacy of the new therapeutic or the progression of CTCL or CTCL with blood involvement in the subject prior to, during, or after the administration of the new therapy.

### Kits and Detecting Devices for Measuring Biomarkers

The present disclosure also provides kits and detecting devices for use in measuring the gene copy number of a biomarker set as described herein. Such a kit or detecting device can comprise one or more binding agents (e.g., probes) that specifically bind to the target biomarkers, such as those listed in Table 1.

For example, such a kit or detecting device may comprise at least one binding agent that is specific to one protein biomarkers selected from Table 1. In some instances, the kit or detecting device comprises binding agents specific to two or more members of the protein biomarker set described herein. In some embodiments, the kit or detecting device comprises probes such as those listed in Table 2.

Aspects of the present disclosure also provide compositions comprising one or more probes that specifically binds to a nucleic acid sequence of any of the biomarkers (e.g., TP53, MYC, RB1, CDKN2A, ATM, STAT3/5B, ARID1A, ZEB1, FAS, CARD11, or DNMT3A). In some embodiments, the one or more probes is a nucleic acid complementary to the nucleic acid sequence of any one of the biomarkers (e.g., TP53, MYC, RB1, CDKN2A, ATM, STAT3/5B, ARID1A, ZEB1, FAS, CARD11, or DNMT3A). Examples of probe sequences are provided in Table 2 and Table 3.

In some embodiments, the one or more probes comprises a detectable label. The detectable label may be a fluorophore. Examples of fluorophores include, without limitation, xanthene derivatives (e.g., fluorescein, rhodamine, Oregon green, eosin and Texas red), cyanine derivatives (e.g., cyanine, indocarbocyanine, oxacarbocyanine, thiacarbocyanine and merocyanine), naphthalene derivatives (e.g., dansyl and prodan derivatives), coumarin derivatives, oxadiazole derivatives (e.g., pyridyloxazole, nitrobenzoxadiazole and benzoxadiazole), pyrene derivatives (e.g., cascade blue), oxazine derivatives (e.g., Nile red, Nile blue, cresyl violet and oxazine 170), acridine derivatives (e.g., proflavin, acridine orange and acridine yellow), arylmethine derivatives (e.g., auramine, crystal violet and malachite green), and tetrapyrrole derivatives (e.g., porphin, phthalocyanine and bilirubin). Other detectable labels may be used in accordance with the present disclosure, such as, for example, gold nanoparticles or other detectable particles or moieties.

In some embodiments, one or more of the binding agents is an antibody that specifically binds to a protein of the biomarker set. In some embodiments, the one or more binding agents is an aptamer, such as a peptide aptamer or oligonucleotide aptamer, that specifically binds to a protein of the biomarker set.

In some embodiments, the kits further comprise a detection agent (e.g., an antibody binding to the binding agent) for detecting binding of the agent to the protein(s) of the biomarker set. The detection agent can be conjugated to a label. In some embodiments, the detection agent is an antibody that specifically binds to at least one of the binding agents. In some embodiments, the binding agent comprises a tag that can be identified and, directly or indirectly, bound by a detection agent.

In the kit or detecting device, one or more of the binding agents may be immobilized on a support member, e.g., a membrane, a bead, a slide, or a multi-well plate. Selection of an appropriate support member for the assay will depend on various factor such as the number of samples and method of detecting the signal released from label conjugated to the agent.

In some embodiments, the support member is a membrane, such as a nitrocellulose membrane, a polyvinylidene fluoride (PVDF) membrane, or a cellulose acetate membrane. In some examples, the assay may be in a Western blot assay format or a nucleic acid microarray assay format.

In some embodiments, the support member is a multi-well plate, such as an ELISA plate. In some embodiments, the immunoassays described herein can be carried out on high throughput platforms. In some embodiments, multi-well plates, e.g., 24-, 48-, 96-, 384- or greater well plates, may be used for high throughput immunoassays. Individual immunoassays can be carried out in each well in parallel. Therefore, it is generally desirable to use a plate reader to measure multiple wells in parallel to increase assay throughput. In some embodiments, plate readers that are capable of imaging multi-wells (e.g., 4, 16, 24, 48, 96, 384, or greater wells) in parallel can be used for this platform. For example, a commercially available plate reader (e.g., the plate vision system available from Perkin Elmer, Waltham, Mass.) may be used. This plate reader is capable of kinetic-based fluorescence analysis. The plate vision system has high collection efficiency optics and has special optics designed for the analysis of 96 wells in parallel. Additional suitable parallel plate readers include but are not limited to the SAFIRE (Tecan, San Jose, Calif.), the FLIPRTETRA® (Molecular Devices, Union City, Calif.), the FDSS7000 (Hamamatsu, Bridgewater, N.J.), and the CellLux (Perkin Elmer, Waltham, Mass.).

The kit can also comprise one or more buffers as described herein but not limited to a coating buffer, a blocking buffer, a wash buffer, and/or a stopping buffer.

In some embodiments, the kit can comprise instructions for use in accordance with any of the methods described herein. The included instructions can comprise a description of how to use the components contained in the kit for measuring the amount of a biomarker set (e.g., protein or nucleic acid) in a biological sample collected from a subject, such as a human patient. The instructions relating to the use of the kit generally include information as to the amount of each component and suitable conditions for performing the assay methods described herein. The components in the kits may be in unit doses, bulk packages (e.g., multi-dose packages), or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

The label or package insert indicates that the kit is used for evaluating the level of a biomarker set. Instructions may be provided for practicing any of the methods described herein.

The kits of this present disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an PCR machine, a nucleic acid array, or a flow cytometry system.

Kits may optionally provide additional components such as interpretive information, such as a control and/or standard or reference sample. The kit can comprise a container and a label or package insert(s) on or associated with the container. In some embodiments, the present disclosure provides articles of manufacture comprising contents of the kits described above.

### Treatment of Cutaneous T Cell Lymphoma (CTCL)

A subject having or at risk for CTCL or CTCL with blood involvement, as identified using the methods described herein, may be treated with any appropriate therapy. In some embodiments, provided methods include selecting a treatment for a subject based on the output of the described method, e.g., determining the presence or absence of a genetic abnormality in a biomarker or a biomarker set.

In some embodiments, the method comprises one or both of selecting or administering a therapy, e.g., chemotherapy, corticosteroid, radiation, phototherapy, and/or retinoids, for administration to the subject based on the output of the assay, e.g., biomarker detection.

In some embodiments, the therapy comprises administering an chemotherapy. Examples of the chemotherapeutic agents include, but are not limited to, mechlorethamine HCl, methotrexate, pralatrexate, gemcitabine, pentostatin, liposomal doxorubicin, chlorambucil, cyclophosphamide, etoposide, temozolomide, alemtuzumab, denileukin diftitox, vorinostat, and romidepsin.

Additional examples of chemotherapy include, but are not limited to, Platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin HCl, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives); Purine antagonists (Thioguanine, Fludarabine, Cladribine, 6-Mercaptopurine, Pentostatin, clofarabine and relatives) and Pyrimidine antagonists (Cytarabine, Floxuridine, Azacitidine, Tegafur, Carmofur, Capacitabine, Gemcitabine, hydroxyurea, 5-Fluorouracil (5FU), and relatives); Alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives); nitrosoureas (e.g., Carmustine, Lomustine, Semustine, Fotemustine, Nimustine, Ranimustine, Streptozocin, and relatives); Triazenes (e.g., Dacarbazine, Altretamine, Temozolomide, and relatives); Alkyl sulphonates (e.g., Busulfan, Mannosulfan, Treosulfan, and relatives); Procarbazine; Mitobronitol, and Aziridines (e.g., Carboquone, Triaziquone, ThioTEPA, triethylenemalamine, and relatives); Antibiotics, such as Hydroxyurea, Anthracyclines (e.g., doxorubicin agent, daunorubicin, epirubicin and other derivatives); Anthracenediones (e.g., Mitoxantrone and relatives); Streptomyces family (e.g., Bleomycin, Mitomycin C, Actinomycin, Plicamycin); and Ultraviolet light.

In some embodiments, the therapy comprises administering a corticosteroid. Examples of corticosteroids include, but are not limited to, hydrocortisone, methylprednisolone, prednisolone, prednisone, triamcinolone, amcinonide, budesonide, desonide, fluocinolone acetonide, fluocinonide, halcinonide, triamcinolone acetonide, beclometasone, betamethasone, dexamethasone, fluocortolone, halometasone, mometasone, alclometasone dipropionate, betamethasone dipropionate, betamethasone valerate, clobetasol propionate, clobetasone butyrate, fluprednidene acetate, mometasone furoate, ciclesonide, cortisone acetate, hydrocortisone aceponate, hydrocortisone acetate, hydrocortisone buteprate, hydrocortisone butyrate, hydrocortisone valerate, prednicarbate, and tixocortol pivalate.

In some embodiments, the therapy comprises administering radiation therapy. Examples of radiation therapy include, but are not limited to, ionizing radiation, gamma-radiation, neutron beam radiotherapy, electron beam radiotherapy, proton therapy, systemic radioactive isotopes, and radiosensitizers.

In some embodiments, the therapy comprises administering a phototherapy. Examples of phototherapy include, but are not limited to, broad-band UVB therapy, narrow-band UVB therapy, psoralen plus ultraviolet A (PUVA) therapy, and UVA1 therapy.

In some embodiments, the therapy comprises administering a retinoid. Examples of retinoids include, but are not limited to, bexarotene, tazarotene, tretinoin, isotretinoin, and alitretinoin.

An effective amount of the CTCL or CTCL with blood involvement therapy can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation, or topical routes.

“An effective amount” as used herein refers to the amount of each active agent that produces a desired therapeutic effect on the subject, either alone or in combination with one or more other active agents. Effective amounts vary, as recognized by those skilled in the art, depending on the particular condition being treated, the severity of the condition, the individual patient parameters including age, physical condition, size, weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations such as the half-life of an agent will generally contribute to the determination of the dosage. Frequency of administration may be determined and adjusted over the course of therapy, and is generally, but not necessarily, based on treatment and/or suppression and/or amelioration and/or delay of CTCL or CTCL with blood involvement. Alternatively, sustained continuous release formulations of therapeutic agent may be appropriate. Various formulations and devices for achieving sustained release are known in the art.

As used herein, the term “treating” refers to the application or administration of a composition including one or more active agents to a subject who has CTCL or CTCL with blood involvement, a symptom of CTCL or CTCL with blood involvement, and/or a predisposition toward CTCL or CTCL with blood involvement, with the purpose to cure, heal, alleviate, relieve, alter, remedy, ameliorate, improve, or affect the disorder, the symptom of the disease, and/or the predisposition toward CTCL or CTCL with blood involvement.

Alleviating CTCL or CTCL with blood involvement includes delaying the development or progression of the disease, and/or reducing disease severity. Alleviating the disease does not necessarily require curative results.

As used herein, “delaying” the development of a disease (such as CTCL or CTCL with blood involvement) means to defer, hinder, slow, retard, stabilize, and/or postpone progression of the disease. This delay can be of varying lengths of time, depending on the history of the disease and/or individuals being treated. A method that “delays” or alleviates the development of a disease and/or delays the onset of the disease is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of CTCL or CTCL with blood involvement includes initial onset and/or recurrence.

In some embodiments, the therapy is administered one or more times to the subject. The therapy, e.g., chemotherapy, corticosteroid, radiation, phototherapy, and/or retinoids, may be administered along with another therapy as part of a combination therapy for treatment of CTCL or CTCL with blood involvement.

The term combination therapy, as used herein, embraces administration of these agents in a sequential manner, that is, wherein each therapeutic agent is administered at a different time, as well as administration of these therapeutic agents, or at least two of the agents, in a substantially simultaneous manner.

Sequential or substantially simultaneous administration of each agent can be affected by any appropriate route including, but not limited to, oral routes, intravenous routes, intramuscular, subcutaneous routes, and direct absorption through mucous membrane tissues. The agents can be administered by the same route or by different routes. For example, a first agent can be administered orally, and a second agent can be administered intravenously.

As used herein, the term “sequential” means, unless otherwise specified, characterized by a regular sequence or order, e.g., if a dosage regimen includes the administration of a first therapeutic agent and a second therapeutic agent, a sequential dosage regimen could include administration of the first therapeutic agent before, simultaneously, substantially simultaneously, or after administration of the second therapeutic agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the agents described herein.

Without further elaboration, it is believed that one skilled in the art can, based on the above description, utilize the present invention to its fullest extent. The following specific embodiments are, therefore, to be construed as merely illustrative, and not limiting. All publications cited herein are incorporated by reference for the purposes or subject matter referenced herein.

### EXAMPLES

### Example 1: Abnormal Gene Copy Number was Detected in Blood Samples from Patients with L-CTCL Using the 11 Probe FISH Panel

### Materials and Methods

### Isolation of Malignant T Cells in Leukemic CTCL

Approximately 45 mL of peripheral blood was obtained from each patient in Sodium Heparin Blood Collection Tubes (BD Vacutainer) and diluted with 90 mL PBS. Diluted blood was layered over 15 mL of ficoll (GE Healthcare Ficoll-Paque Premium or Isolymph from CTL Scientific Supply Corp.) in 50 mL conical tubes prior to centrifugation for 35 minutes at 1500 rpm. Buffy coats were isolated and washed with RPMI in 15 mL conical tubes, centrifuged for 10 minutes at 1400 rpm, resuspended in RPMI, rewashed in RPMI and centrifuged again for 8 minutes at 1100 rpm to minimize residual Ficoll. After RPMI resuspension, cells were counted (and if not immediately processed further were frozen in 90% RPMI 10% DMSO in liquid nitrogen). Malignant T cells were isolated from total mononuclear cells by either fluorescence-activated cell sorting (FACS) or magnetic bead sorting.

For samples sorted by FACS, mononuclear cells were first stained with: anti-CD3-BV-421 (clone OKT3, Biolegend, San Diego, Calif.), anti-CD4-PerCP (clone RPA-T4, Biolegend), anti-CD7-APC (clone CD7-6B7, Biolegend), anti-CD8-PECy7 (clone SK1, Biolegend), anti-CD26-PE (clone BA5b, Biolegend). For patients with a known V-beta clone, cells were also stained with corresponding anti-Vbeta-PE (Beckman Coulter, Inc.). After washing twice with stain buffer, cells were sorted (BD FACSAria) with gates to identify population previously defined as abnormal by clinical flow cytometry. In most cases, lymphocytes were gated from forward- and side-scatter followed by subsequent gating on CD3+CD4+CD8− cells to allow visualization of CD4+ T cells. (If V-beta antibody was present, V-beta positive CD4+ T cells were further gated thereafter.) A scatter plot of CD4+(and V-beta+ if present) T cells along CD26 and CD7 axes allowed for sorting of the previously defined abnormal populations, whether CD26-CD7-, CD26-CD7+/−, or CD26+/−CD7-. In cases where previous studies demonstrated an abnormal population with atypical markers, such as CD3 dim or CD4 dim, corresponding adjustments in gating were made. For controls, CD3+CD8+ lymphocytes were sorted simultaneously.

For samples sorted by magnetic beads, mononuclear cells were stained with T Cell Biotin-Antibody Cocktail as well as anti-CD26-biotin (eBioscience) and/or anti-CD7-biton (eBioscience) and incubated with anti-biotin microbeads according to the manufacturer's instructions to isolate populations previously identified as abnormal by flow cytometry; in most cases this population was CD4+CD26−CD7− or CD4+CD26−CD7+/−.

### Fixation

Sorted cells were pelleted in a 5 ml centrifuge tube and gently resuspended in 4.5 ml hypotonic solution (0.075M KCl). After 16 minutes incubation at room temperature, 300 ul fresh fixative (3 parts methanol to 1 part acetic acid) was added to each sample, followed by gentle inversion and 10 minutes incubation at room temperature. Next cells were centrifuged at 1100 rpm for 10 minutes, resuspended in 4 ml fixative, incubated at room temperature for 10 minutes, and centrifuged again at 1100 rpm for 10 minutes. After resuspension in 1 ml fixative, samples were stored at 34 for up to 1 month before performing FISH.

### Hybridization

For both commercially available and custom probes, fixed samples underwent overnight FISH hybridization using probes for TP53, ATM, MYC, RB, and CDKN2A (P53/ATM Probe Combination LPH 052, cMYC Breakapart LPH 010, RB1 Deletion LPS 011, P16 Deletion LPH 009-A; Cytocell Aquarius) and newly developed probes for ARID1A, ZEB1, STAT3/5B, DNMT3A, CARD11, and FAS (Cytocell myProbes Custom Probes). Hybridization for all probes was performed according to standard manufacturer instructions for CytoCell Aquarius probes.

### Probe Visualization and Quantification

Probes were quantified using a fluorescent light microscope (BX-60 or BX-43, Olympus; or Axio Observer Z1, Zeiss) running CytoVision (Version 7.4, Leica Biosystems) or TissueFAXs (Version 4.2, TissueGnostics) software. For each probe, signals in 100 or 200 nuclei were examined.

### Probe Sequences

Sequences can be obtained from the referenced nucleotide code start and end sequence position numbers as given in Table 2 and accessed in the readily available database Gencode Ensembl v74 (GRCh37). Commercially available probes used in this study are provided in Table 3.

TABLE 3
Commercially available probes.

| Gene Probe | Source |
|---|---|
| TP53 Probe | cytocellus.com/assets/000/001/718/ |
| | LPD504ASR_v001.00_original.pdf?1466589135 |
| p53/ATM Probe | cytocell-us.com/assets/000/001/248/ |
| Combination | LPH052ASR_v005.00_original.pdf?1437574348 |
| cMYC | cytocell-us.com/assets/000/001/210/ |
| Breakapart Probe | LPH010ASR_v009.00_original.pdf?1437574184 |
| RB1 Deletion | cytocell-us.com/assets/000/001/311/ |
| Probe | LPS011_ASR_v007.00_original.pdf?1437574599 |
| p16 Deletion | cytocell-us.com/assets/000/001/209/ |
| Probe | LPH009ASR_v008.00_original.pdf?1437574181 |

### Patient Characteristics

As described here, genetic abnormalities in sorted or unsorted peripheral blood from 24 patients was assessed using the 11-probe panel. Patient characteristics are provided in the tables below.

TABLE 4
FISH signal number (reported as 2 if no abnormal population over cutoff).
Fish signal number (reported as 2 if no abnormal population over cutoff)

| Patient | | | | | | | | | | | |
|---|---|---|---|---|---|---|---|---|---|---|---|
| ID | TP53 | ARID1A | ZEB1 | CDKN2A | DNMT3A | ATM | RB1 | FAS | MYC | STAT3/5B | CARD11 |
| 1 | 1 | 2 | 2 | 2 | 2 | 2 | 2 | 2 | 4 | 2 | 2 |
| 2 | 1 | 1 | 2 | 2 | 2 | 2 | 2 | 1 | 2 | 3 | 2 |
| 3 | 1 | 2 | 2 | 1 | 2 | 1 | 2 | 2 | 3 | 3 | 2 |
| 4 | 1 | 1 | 1 | 2 | 2 | 2 | 2 | 2 | 3 | 3 | 2 |
| 5 | 1 | 2 | 1 | 1 | 1 | 2 | 2 | 1 | 2 | 2 | 2 |
| 6 | 1 | 1 | 1 | 2 | 1 | 2 | 2 | 2 | 2 | 3 | 1 |
| 7 | 1 | 2 | 0 | 2 | 1 | 2 | 1 | 2 | 3 | 3 | 3 |
| 8 | 1 | 1 | 1 | 1 | 1 | 2 | 1 | 1 | 4 | 2 | 2 |
| 9 | 1 | 2 | 1 | 2 | 1 | 2 | 2 | 1 | 3 | 3 | 3 |
| 10 | 1 | 4 | 2 | 1 | 4 | 2 | 4 | 4 | 4 | 3.6 | 5 |
| 11 | 1 | 2 | 2 | 1 | 2 | 1 | 1 | 2 | 2 | 3 | 2 |
| 12 | 1 | 1 | 2 | 2 | 2 | 1 | 2 | 2 | 3 | 3 | 3 |
| 13 | 1 | 1 | 2 | 2 | 1 | 2 | 2 | 2 | 3 | 3 | 2 |
| 14 | 2 | 2 | 2 | 2 | 2 | 2 | 2 | 2 | 3 | 1 | 2 |
| 15 | 2 | 2 | 2 | 2 | 2 | 2 | 2 | 2 | 2 | 2 | 2 |
| 16 | 2 | 2 | 2 | 2 | 2 | 2 | 2 | 2 | 2 | 2 | 2 |
| 17 | 2 | 2 | 2 | 2 | 2 | 2 | 2 | 2 | 2 | 2 | 2 |
| 18 | 2 | 2 | 2 | 2 | 2 | 2 | 2 | 2 | 2 | 2 | 2 |
| 19 | 2 | 2 | 2 | 2 | 2 | 2 | 2 | 2 | 2 | 2 | 2 |
| 20 | 2 | 2 | 2 | 2 | 2 | 2 | 2 | 2 | 2 | 2 | 2 |
| 21 | 2 | 2 | 2 | 2 | 2 | 2 | 2 | 2 | 2 | 2 | 2 |
| 22 | 2 | 2 | 2 | 2 | 2 | 2 | 2 | 2 | 2 | 2 | 2 |
| 23 | 2 | 2 | 2 | 2 | 2 | 2 | 2 | 2 | 2 | 2 | 2 |
| 24 | 2 | 2 | 2 | 2 | 2 | 2 | 2 | 2 | 2 | 2 | 2 |

TABLE 5
FISH signal number reported as percentage of total cells with FISH signal.

| Patient | Percentage of total cells with FISH signal number | | | | | | | | | | |
|---|---|---|---|---|---|---|---|---|---|---|---|
| ID | TP53 | ARID1A | ZEB1 | CDK2NA | DNMT3A | ATM | RB1 | FAS | MYC | STAT3/B | CARD11 |
| 1 | 85.0% | 100.0% | 100.0% | 99.0% | 100.0% | 100.0% | 100.0% | 100.0% | 86.0% | 100.0% | 100.0% |
| 2 | 88.0% | 42.5% | 42.5% | 98.0% | 100.0% | 88.0% | 99.0% | 88.0% | 98.0% | 42.5% | 100.0% |
| 3 | 100.0% | 100.0% | 100.0% | 98.0% | 100.0% | 100.0% | 100.0% | 100.0% | 97.0% | 88.5% | 100.0% |
| 4 | 76.0% | 58.5% | 58.5% | 95.5% | 98.0% | 100.0% | 100.0% | 98.0% | 80.0% | 58.5% | 98.0% |
| 5 | 34.5% | 100.0% | 11.0% | 28.5% | 36.0% | 100.0% | 99.0% | 36.0% | 94.0% | 100.0% | 100.0% |
| 6 | 94.0% | 84.0% | 61.0% | 99.0% | 92.0% | 94.0% | 100.0% | 92.0% | 100.0% | 23.0% | 92.0% |
| 7 | 73.5% | 100.0% | 64.0% | 99.0% | 94.0% | 100.0% | 71.0% | 100.0% | 50.6% | 64.0% | 94.0% |
| 8 | 82.0% | 54.0% | 54.0% | 36.0% | 36.0% | 100.0% | 87.0% | 36.0% | 13.5% | 100.0% | 100.0% |
| 9 | 59.9% | 100.0% | 64.0% | 100.0% | 47.0% | 100.0% | 100.0% | 59.0% | 59.0% | 46.0% | 59.0% |
| 10 | 53.0% | 39.5% | 100.0% | 87.0% | 40.5% | 100.0% | 36.0% | 40.5% | 15.0% | 52%, 39.5% | 40.5% |
| 11 | 40.0% | 90.0% | 90.0% | 56.0% | 100.0% | 70.0% | 64.0% | 100.0% | 100.0% | 90.0% | 100.0% |
| 12 | 99.5% | 94.0% | 96.0% | 100.0% | 99.5% | 99.5% | 100.0% | 92.0% | 52.0% | 80.0% | 99.5% |
| 13 | 95.0% | 80.5% | 100.0% | 100.0% | 91.0% | 95.0% | 99.5% | 100.0% | 80.5% | 80.5% | 100.0% |
| 14 | 100.0% | 100.0% | 100.0% | 99.5% | 100.0% | 100.0% | 100.0% | 100.0% | 62.0% | 9.0% | 100.0% |
| 15 | 100.0% | 100.0% | 100.0% | 98.5% | 100.0% | 100.0% | 100.0% | 100.0% | 93.0% | 100.0% | 94.5% |
| 16 | 100.0% | 99.0% | 99.0% | 100.0% | 100.0% | 100.0% | 99.0% | 100.0% | 99.0% | 99.0% | 100.0% |
| 17 | 100.0% | 100.0% | 100.0% | 100.0% | 100.0% | 100.0% | 100.0% | 100.0% | 100.0% | 100.0% | 100.0% |
| 18 | 100.0% | 99.5% | 99.5% | 99.5% | 99.5% | 100.0% | 99.5% | 100.0% | 99.5% | 99.5% | 99.5% |
| 19 | 99.0% | 100.0% | 100.0% | 100.0% | 100.0% | 99.0% | 100.0% | 100.0% | 100.0% | 100.0% | 100.0% |
| 20 | 99.0% | 100.0% | 100.0% | 100.0% | 100.0% | 99.0% | 100.0% | 100.0% | 100.0% | 100.0% | 100.0% |
| 21 | 99.0% | 100.0% | 100.0% | 98.5% | 98.5% | 99.0% | 99.5% | 98.5% | 99.5% | 100.0% | 98.5% |
| 22 | 100.0% | 100.0% | 100.0% | 100.0% | 99.5% | 100.0% | 100.0% | 99.5% | 100.0% | 100.0% | 99.5% |
| 23 | 100.0% | 100.0% | 100.0% | 99.0% | 100.0% | 100.0% | 99.0% | 100.0% | 100.0% | 100.0% | 100.0% |
| 24 | 100.0% | 100.0% | 100.0% | 99.0% | 99.0% | 100.0% | 100.0% | 99.0% | 99.5% | 100.0% | 99.0% |

TABLE 6
Patient characteristics.

| | Patient characteristics | | | | | | | | |
|---|---|---|---|---|---|---|---|---|---|
| | | | 2007 | | | | | | |
| | | | ISCL | | | | Percent | Percent | |
| Patient | | Age | blood | | | CD3+ | CD26− of | CD7− | Sorted |
| ID | Diagnosis | Sex | level | PCR | CD4/CD8 | T Cell/μl | CD4+ | of CD4+ | population |
| 1 | Sezary | 69 M | B2 | + | 5.8 | 534 | 31.0% | 33.8% | CD3 + CD4 + |
| | | | | | | | | | CD7 − CD26 − |
| 2 | Sezary | 69 F | B2 | + | 13.5 | 2937 | 67.8% | 98.0% | CD3 + CD4 + |
| | | | | | | | | | CD7 + CD26 − |
| 3 | Sezary | 63 F | B2 | − | 8.6 | 533 | 19.1% | 42.2% | CD3 + CD4 + |
| | | | | (Vbeta+) | | | | | CD26 − VB7.2 + |
| 4 | Sezary | 79 F | B2 | + | 25.9 | 757 | 46.5% | 3.8% | CD3 + CD4 + |
| | | | | | | | | | CD7 − CD26 − VB3 + |
| 5 | Sezary | 91 M | B2 | + | 6.1 | 1362 | 44.9% | 50.7% | CD3 + CD4 + |
| | | | | | | | | | CD7 − CD26 − |
| 6 | Sezary | 87 M | B2 | + | 3.6 | 454 | 40.4% | 16.5% | CD3 + CD4 + |
| | | | | | | | | | CD26 − |
| 7 | Sezary | 73 F | B2 | + | 25.6 | 20235 | 90.3% | 88.4% | non sorted |
| | | | | | | | | | PBMC |
| 8 | Sezary | 63 F | B2 | + | 6.6 | 838 | 63.8% | 9.4% | CD3 + CD4 + |
| | | | | | | | | | CD7 + CD26 − |
| 9 | Sezary | 76 M | B2 | + | 58.5 | 9764 | 90.7% | 86.7% | non sorted |
| | | | | | | | | | PBMC |
| 10 | Sezary | 82 M | B2 | + | 10.4 | 3083 | 52.7% | 62.7% | CD3 + CD4 + |
| | | | | | | | | | CD26 − VB2 + |
| 11 | Follicular MF | 72 M | B0 | + | 8.4 | 1437 | 14.1% | 19.7% | CD3 + CD4 + |
| | | | | | | | | | CD7 − CD26 − VB20 + |
| 12 | MF with tumor | 61 M | B2 | + | 16.7 | 2967 | 29.8% | 14.8% | CD3 + CD4 + |
| | | | | | | | | | CD26 − |
| 13 | Patch/plaque MF | 60 M | B2 | + | 4.1 | 484 | 14.9% | 53.0% | CD3dimCD4 + |
| | | | | | | | | | CD7 + CD26 − |
| 14 | Patch/plaque MF | 51 M | B0 | + | 2.7 | 1141 | 29.8% | 14.6% | CD3 + CD4 + |
| | | | | | | | | | CD26 − |
| 15 | Follicular MF | 47 M | B0 | + | 1.1 | 553 | 5.0% | 5.8% | VB2 + |
| 16 | Patch/plaque MF | 61 F | B0 | + | 2.7 | 1415 | 10.4% | 27.3% | CD3 + CD4 + |
| | | | | | | | | | CD7 − CD26 − |
| 17 | Patch/plaque MF | 82 F | B2 | + | 2.9 | 1071 | 21.8% | 41.5% | CD3 + CD4 + |
| | | | | | | | | | CD7 − CD26 − |
| 18 | Patch/plaque MF | 86 M | B0 | + | 1.5 | 1110 | 20.8% | 18.8% | CD3 + CD4 + |
| | | | | | | | | | CD26 − |
| 19 | Patch/plaque MF | 76 M | B0 | + | 2.6 | 820 | 11.2% | 14.6% | CD3 + CD4 + |
| | | | | | | | | | CD7 − CD26 − |
| 20 | Patch/plaque MF | 66 F | B0 | NA | 4.0 | 713 | 9.4% | 6.1% | CD3 + CD4 + |
| | | | | | | | | | CD7 − CD26 − |
| 21 | Patch/plaque MF | 84 M | B0 | + | 1.5 | 1478 | 22.5% | 18.0% | CD3 + CD4 + |
| | | | | | | | | | CD7 − CD26 − |
| 22 | Follicular | 37 M | B0 | − | 5.7 | 1493 | 23.4% | 15.6% | CD3 + CD4 + |
| | mucinosis | | | | | | | | CD26 − |
| 23 | Patch/plaque MF | 74 F | B0 | NA | 1.9 | 505 | 17.0% | 15.2% | CD3 + CD4 + |
| | | | | | | | | | CD26 − |
| 24 | Angioedematous | 73 F | B0 | + | 1.3 | 367 | 23.6% | 15.9% | CD3 + CD4 + |
| | plaques | | | | | | | | CD26 − |

### Results

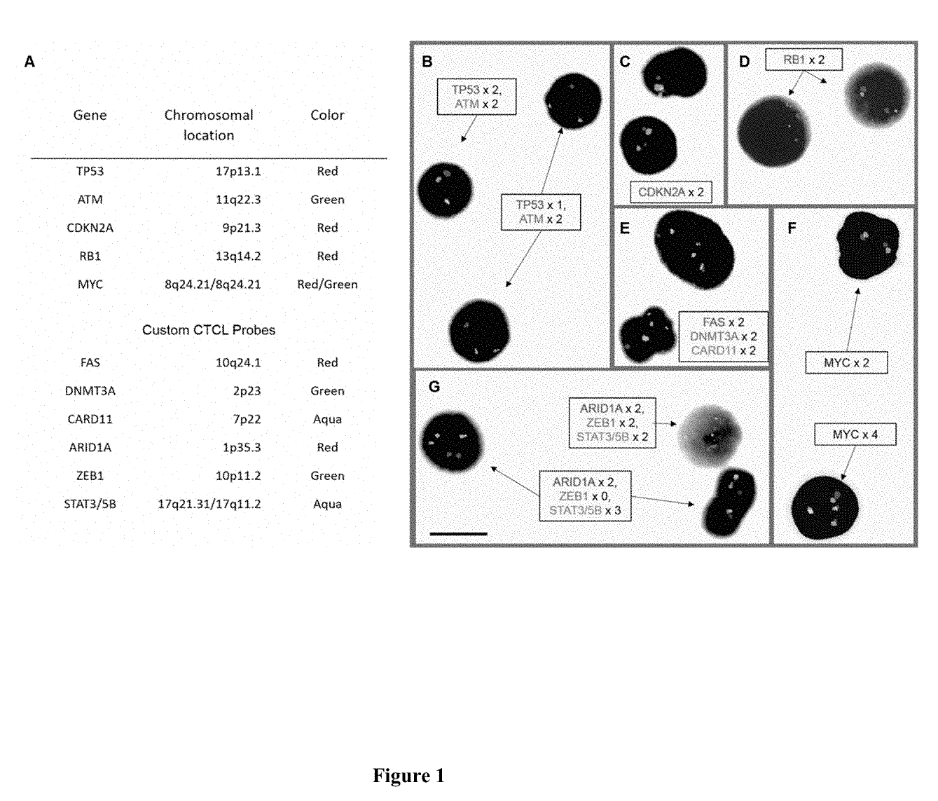

After design and production of FISH probes for STAT3/5B, ARID1A, ZEB1, FAS, and CARD11, probe hybridization sites were validated on metaphase chromosome spreads. FIG. 1, panel A shows genes, chromosomal locations and fluorescent colors for the 11 clinically validated probes used in the CTCL FISH panel.

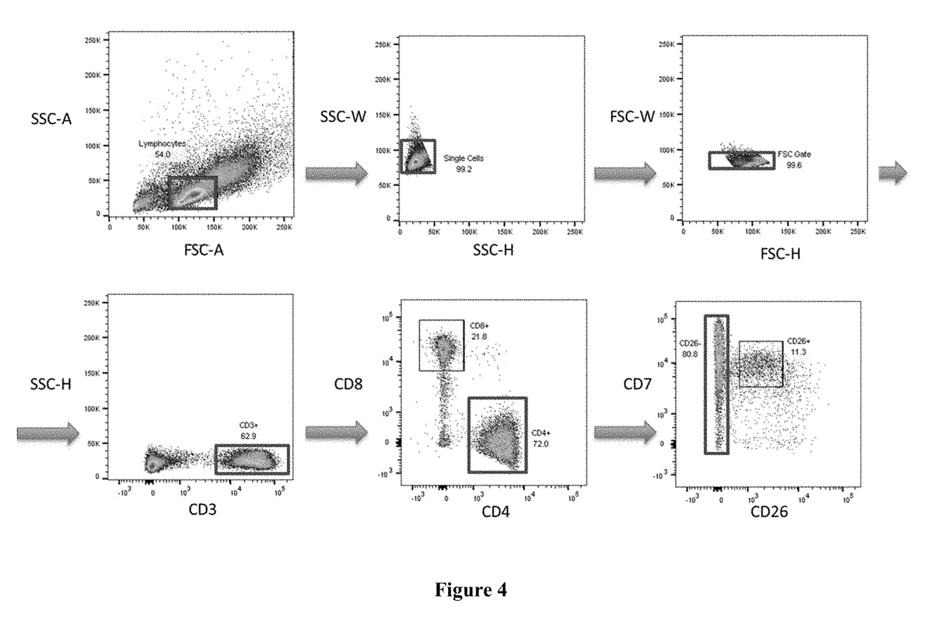

Written informed consent of 24 patients who had a confirmed or suspected diagnosis of CTCL was obtained in accordance with protocols approved by the Institutional Review Board of Yale School of Medicine. Populations enriched for abnormal CD3+CD4+ lymphocytes, most frequently also CD26− and/or CD7−, were purified from Ficoll-isolated PBMC by either flow cytometric sorting or by magnetic bead isolation (FIG. 4). Samples underwent fixation and overnight FISH hybridization with the panel of 11 probes, and probe signals were quantified by fluorescence light microscopy at 100 or 200 nuclei per probe (FIG. 1, panel B). CD8+ T cells collected as control populations were used to generate cutoffs (Table 7) for positive FISH abnormalities at the 99^{th} percentile of binomial distribution of proportions calculated for each probe (Wiktor et al., 2006).

TABLE 7
CD8+ sorted cells: percentage of cells with normal (2x) signal of total counted (100 or
200) cells.

| Patient ID | TP53 | ATM | MYC | RB1 | CDKN2A | ARID1A | ZEB1 | STAT3/5B | FAS | DMNT3A | CARD11 |
|---|---|---|---|---|---|---|---|---|---|---|---|
| 1 | 97 | 98 | 93 | 92 | 84 | 97 | 97 | 94 | 95 | 98 | 98 |
| 2 | 94 | 99 | 94 | 95 | 94 | 94 | 97 | 97 | 98 | 97 | 95 |
| 3 | 99 | 98 | 96 | 100 | 97 | 98 | 94 | 96 | 97 | 97 | 98 |
| 4 | 97 | 96 | 97 | 97 | 99 | 94 | 98 | 99 | 97 | 97 | 99 |
| 8 | 97 | 96 | 95 | 99 | 95 | 97 | 97 | 96 | 94 | 98 | 98 |
| 11 | 96 | 95 | 99 | 94 | 97 | 97 | 100 | 93 | 99 | 99 | 98 |
| 15 | 96 | 95 | 94 | 91 | 94 | 96 | 95 | 99 | 98 | 99 | 100 |
| 18 | 98 | 96 | 93 | 96 | 96.5 | 94 | 96 | 97 | 97 | 97 | 99 |
| Mean | 96.750 | 96.625 | 95.125 | 95.500 | 94.563 | 95.875 | 96.750 | 96.375 | 96.875 | 97.750 | 98.125 |
| STDEV | 1.488 | 1.506 | 2.100 | 3.162 | 4.594 | 1.642 | 1.832 | 2.134 | 1.642 | 0.886 | 1.458 |
| 99.00% | 92.286 | 92.107 | 88.824 | 86.013 | 80.781 | 90.949 | 91.253 | 89.973 | 91.949 | 95.091 | 93.752 |
| (M+/−3STDV)% | 46.143 | 46.054 | 44.412 | 43.007 | 40.391 | 45.474 | 45.627 | 44.987 | 45.974 | 47.545 | 46.876 |
| False Pos # | 3.250 | 3.375 | 4.875 | 4.500 | 5.438 | 4.125 | 3.250 | 3.625 | 3.125 | 2.250 | 1.875 |
| Normal 95% Cutoff | 0.077 | 0.078 | 0.096 | 0.092 | 0.102 | 0.087 | 0.077 | 0.081 | 0.075 | 0.064 | 0.059 |
| Normal 99% Cutoff | 0.098 | 0.099 | 0.119 | 0.114 | 0.125 | 0.109 | 0.098 | 0.103 | 0.096 | 0.084 | 0.078 |

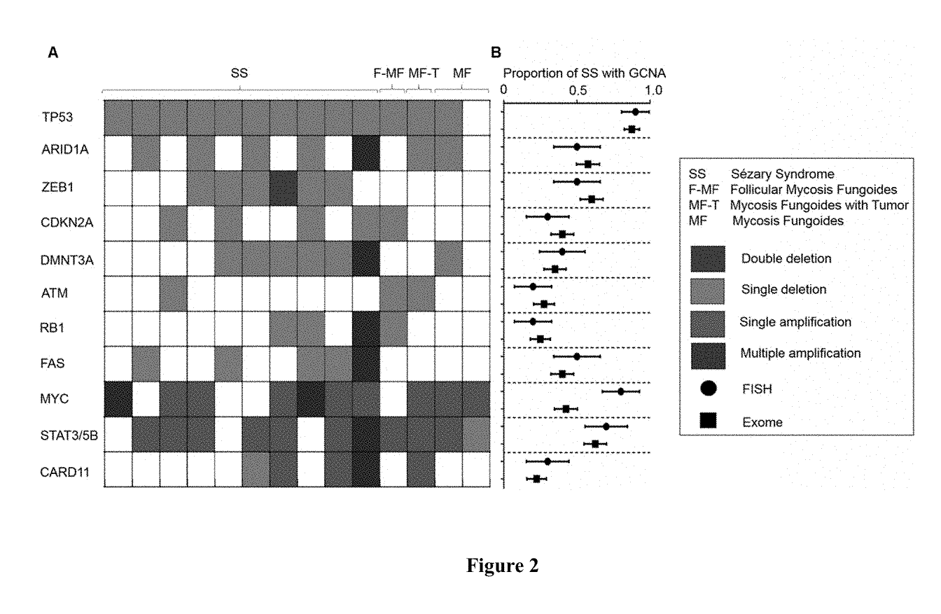

GCNAs were detected by FISH (FIG. 2, panel A) in 10 of 10 patients meeting International Society of Cutaneous Lymphoma (ISCL) criteria for SS and B2 stage blood involvement, 1 patient with F-MF not meeting B2 criteria, 1 patient with tumor-stage MF meeting B2 criteria, and 2 patients with MF one of whom met B2 criteria. No abnormalities were detected by FISH in the other 10 patients, of whom only 1 patient (with MF) met B2 involvement by one criterion (41.5% CD7− of CD4+ T cells; sorted CD7− cells). Among the 9 remaining patients with no detected GCNAs, 6 had a diagnosis of MF; 1 had a diagnosis of F-MF; 1 had a diagnosis of follicular mucinosis in association with Crohn's disease (elevated CD4/8 ratio of 5.7, negative T cell receptor (TCR) PCR analysis for clonality); and 1 had atypical angioedematous plaques with initially positive (but subsequently negative) TCR clonality by PCR. Overall, of patients meeting revised 2007 ISCL criteria for B2 blood involvement, 12 of 13 patients (92%) had GCNAs detected by FISH.

These results demonstrated that abnormal gene copy numbers were detected in blood samples from patients with L-CTCL using the 11 probe FISH panel.

Proportions of FISH-identified GCNAs present per gene in patients with Sézary syndrome did not significantly differ from those observed in a recent large-scale exome study on a separate patient cohort (p-values 0.27-1.00, Fisher's exact test, FIG. 2, panel B, Table 8). One patient had an unanticipated single deletion of CARD11 in 92% of cells scored. CARD11 promotes the T cell receptor-mediated activation cascade and has been found amplified in CTCL exome data (Choi et al., 2015). However separate comparative genomic hybridization (CGH) data have shown a more balanced frequency of amplification and deletion in CARD11 (Kiel et al., 2015), and its functional significance in CTCL cells remains incompletely explored. Multiple atypical amplifications, including 4× amplifications in ARID1A, DNMT3A, RB1, and FAS, were seen in a separate patient with longstanding disease.

TABLE 8
Exome compared to FISH detected gene copy number alterations (GCNAs) in Sézary
syndrome.

| | | | | | | | | | | | | Total |
|---|---|---|---|---|---|---|---|---|---|---|---|---|
| | | | | | | | | | | STAT3/ | | Sezary |
| Genes | TP53 | ARID1A | ZEB1 | CDK2NA | DNMT3A | ATM | RB1 | FAS | MYC | 5B | CARD11 | patients |
| Patients | 35 | 23 | 24 | 16 | 14 | 11 | 10 | 16 | 17 | 25 | 9 | 40 |
| with | | | | | | | | | | | | |
| GCNAs, | | | | | | | | | | | | |
| exome | | | | | | | | | | | | |
| study | | | | | | | | | | | | |
| Proportion | 0.875 | 0.575 | 0.600 | 0.400 | 0.350 | 0.275 | 0.250 | 0.400 | 0.425 | 0.625 | 0.225 | |
| Abnormal | | | | | | | | | | | | |
| Standard | 0.052 | 0.078 | 0.077 | 0.077 | 0.075 | 0.071 | 0.068 | 0.077 | 0.078 | 0.077 | 0.066 | |
| Error | | | | | | | | | | | | |
| Patients | 9 | 5 | 5 | 3 | 4 | 2 | 2 | 5 | 8 | 7 | 3 | 10 |
| with | | | | | | | | | | | | |
| GCNAs, | | | | | | | | | | | | |
| FISH | | | | | | | | | | | | |
| (separate | | | | | | | | | | | | |
| cohort) | | | | | | | | | | | | |
| Proportion | 0.900 | 0.500 | 0.500 | 0.300 | 0.400 | 0.200 | 0.200 | 0.500 | 0.800 | 0.700 | 0.300 | |
| Abnormal | | | | | | | | | | | | |
| Standard | 0.095 | 0.158 | 0.158 | 0.145 | 0.155 | 0.126 | 0.126 | 0.158 | 0.126 | 0.145 | 0.145 | |
| Error | | | | | | | | | | | | |

Within the cohort of patients, specific GCNAs of note include 2 of 10 SS patients with double amplifications of the well-characterized oncogene MYC (Dang, 2012) found within broad amplifications on chromosome 8q in CTCL; joint amplification of several genes has complicated the interpretation of the significance of individual components in this region (Choi et al., 2015). Recent progress on investigational anti-Myc therapeutics (Stellas et al., 2014) may offer a targeted approach to assess the role of this oncogene. Also notable was 1 SS patient with homozygous deletion of ZEB1 and peripheral blood T cell count above 20,000/μL by 8 months after initial clinical presentation with erythroderma. ZEB1, a transcriptional repressor of IL-2 (Wang et al., 2009) and contributor to TGF− β1 mediated growth inhibition in adult T cell leukemia/lymphoma (Nakahata et al., 2010), has been found homozygously deleted in 10% of exome-sequenced L-CTCL patient samples; homozygous deletion in a mouse model has given rise to fatal T cell lymphomas in 84% of affected animals (Hidaka et al., 2008).

The presented 11-probe FISH panel facilitates the diagnosis of CTCL, including L-CTCL and also provides genetic status based on many of the most commonly represented GCNAs reported in CTCL. Many of the genes represented have only recently been published as oncologic drivers of CTCL and these GCNAs are not yet correlated with clinical outcomes. As outcomes data from genetic studies accumulate in the near future, this panel may also provide a helpful tool for prognosis and treatment stratification with advantages including rapid turnaround and ease of clinical implementation in hospitals performing FISH studies. This 11-probe FISH panel can be used to enhance the efficient testing of patients with L-CTC. It suggests the potential utilization of FISH in personalized medicine for CTCL patients.

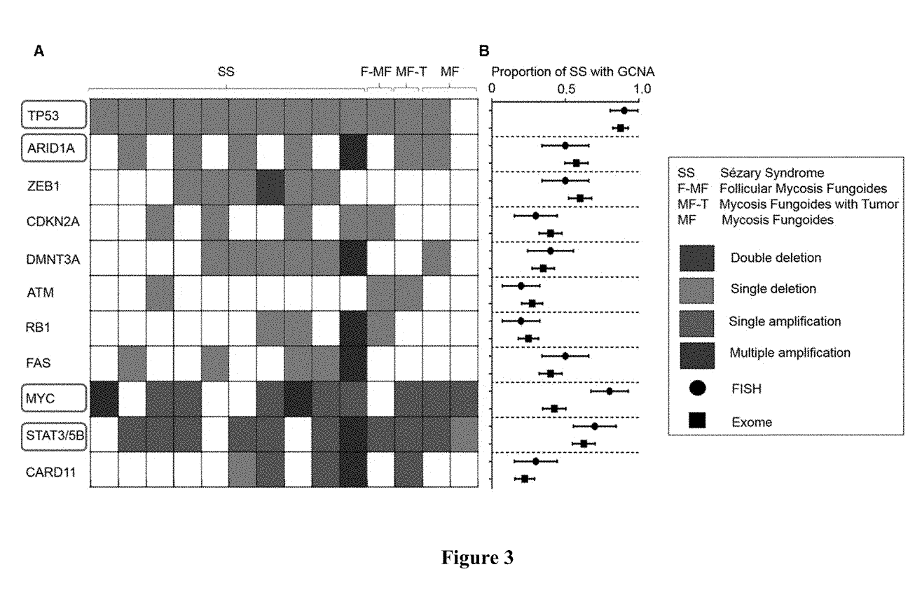

For clinical use, a more practical and cost-effective strategy is to utilize a four probe subset consisting of the two most informative probes assessing deletion (TP53, ARID1A) and amplification (MYC, STAT3/5B) (FIG. 3). Combining exome data (Choi et al., 2015) with the patient cohort, GCNAs have been found present in this subset of genes in 96.2% of CTCL patients with stage B2 blood involvement (two-sided 95% Clopper Pearson confidence interval 87.0%-98.8%).

### REFERENCES

Choi J, Goh G, Walradt T, et al. (2015) Genomic landscape of cutaneous T cell lymphoma. Nat Genet 47:1011-9.
Dang C V (2012) MYC on the path to cancer. Cell 149:22-35.
Gibson J F, Huang J, Liu K J, et al. (2016) Cutaneous T-cell lymphoma (CTCL): Current practices in blood assessment and the utility of T-cell receptor (TCR)-Vβ chain restriction. Journal of the American Academy of Dermatology 74:870-7.
Hidaka T, Nakahata S, Hatakeyama K, et al. (2008) Down-regulation of TCF8 is involved in the leukemogenesis of adult T-cell leukemia/lymphoma. Blood 112:383-93.
Kiel M J, Sahasrabuddhe A A, Rolland D C, et al. (2015) Genomic analyses reveal recurrent mutations in epigenetic modifiers and the JAK-STAT pathway in Sezary syndrome. Nat Commun 6:8470.
Nakahata S, Yamazaki S, Nakauchi H, et al. (2010) Downregulation of ZEB1 and overexpression of Smad7 contribute to resistance to TGF-beta1-mediated growth suppression in adult T-cell leukemia/lymphoma. Oncogene 29:4157-69.
Nuciforo P, Thyparambil S, Aura C, et al. (2016) High HER2 protein levels correlate with increased survival in breast cancer patients treated with anti-HER2 therapy. Mol Oncol 10:138-47.
Stellas D, Szabolcs M, Koul S, et al. (2014) Therapeutic effects of an anti-Myc drug on mouse pancreatic cancer. J Natl Cancer Inst 106.
Van Dyke D L, Werner L, Rassenti L Z, et al. (2016) The Dohner fluorescence in situ hybridization prognostic classification of chronic lymphocytic leukaemia (CLL): the CLL Research Consortium experience. Br J Haematol.
Wang J, Lee S, Teh C E, et al. (2009) The transcription repressor, ZEB1, cooperates with CtBP2 and HDAC1 to suppress IL-2 gene activation in T cells. Int Immunol 21:227-35.
Wiktor A E, Van Dyke D L, Stupca P J, et al. (2006) Preclinical validation of fluorescence in situ hybridization assays for clinical practice. Genet Med 8:16-23.

What is claimed is:

1. A method of detecting a genetic abnormality associated with cutaneous T cell lymphoma (CTCL) or CTCL with blood involvement, comprising:
(a) analyzing a biological sample for a genetic abnormality in TP53, MYC, RB1, CDKN2A, ATM, STAT3/5B, ARID1A, ZEB1, FAS, CARD11, and DNMT3A; and
(b) determining the presence or absence of a genetic abnormality in TP53, MYC, RB1, CDKN2A, ATM, STAT3/5B, ARID1A, ZEB1, FAS, CARD11, and DNMT3A,
wherein if there is a genetic abnormality in at least one of TP53, MYC, RB1, CDKN2A, ATM, STAT3/5B, ARID1A, ZEB1, FAS, CARD11, and DNMT3A, a genetic abnormality associated with CTCL or CTCL with blood involvement is detected.

2. A method of detecting, in a biological sample obtained from an individual, a genetic abnormality associated with cutaneous T cell lymphoma (CTCL) or CTCL with blood involvement, comprising assessing TP53, MYC, RB1, CDKN2A, ATM, STAT3/5B, ARID1A, ZEB1, FAS, CARD11, and DNMT3A for a genetic abnormality, wherein if a genetic abnormality is present in any of/in at least one of TP53, MYC, RB1, CDKN2A, ATM, STAT3/5B, ARID1A, ZEB1, FAS, CARD11, and DNMT3A, the biological sample comprises a genetic abnormality associated with CTCL or CTCL with blood involvement.

3. The method of claim 1, wherein the genetic abnormality is a deletion or an amplification.

4. The method of claim 3, wherein the deletion or abnormality is detected by combining the biological sample with at least one probe selected from ARID1A (Probe 1); ARID1A (Probe 2); ARID1A (Probe 3); CARD11 (Probe 1); CARD11 (Probe 2); CARD11 (Probe 3); ZEB1 (Probe 1); ZEB1 (Probe 2); STAT3/5B (Probe 1); STAT3/5B (Probe 2); STAT3/5B (Probe 3); DNMT3A (Probe 1); DNMT3A (Probe 2); FAS; TP53 Probe; p53/ATM Probe Combination; cMYC Breakapart Probe; RB1 Deletion Probe; and p16 Deletion Probe.

5. A method of determining if an individual has cutaneous T cell lymphoma (CTCL) or CTCL with blood involvement, comprising:
(a) analyzing a biological sample obtained from the individual for a genetic abnormality in at least one of TP53, MYC, RB1, CDKN2A, ATM, STAT3/5B, ARID1A, ZEB1, FAS, CARD11, and DNMT3A;
(b) assessing (enumerating) the copy number of at least one of the 11 genes (TP53, MYC, RB1, CDKN2A, ATM, STAT3/5B, ARID1A, ZEB1, FAS, CARD11, and DNMT3A) in the biological sample;
(c) determining, for each gene for which copy number was assessed, the portion or percent of cells that show an abnormal gene copy number; and
(d) comparing the portion or percent to a reference value or cutoff,
wherein if the portion or percent of cells that show an abnormal gene copy number is greater than the reference value or cutoff, the individual has cutaneous T cell lymphoma (CTCL) or CTCL with blood involvement.

6. The method of 5, wherein the genetic abnormality comprises at least one deletion in at least one of TP53, RB1, CDKN2A, ATM, ARID1A, ZEB1, FAS, and DNMT3A; at least one amplification in at least one of MYC, STAT3/5B, and CARD11; or at least one deletion in at least one of TP53, RB1, CDKN2A, ATM, ARID1A, ZEB1, FAS, and DNMT3A and at least one amplification in at least one of MYC, STAT3/5B, and CARD11.

7. The method of claim 6, wherein if more than about 12% of the population of cells shows an abnormal copy number, the individual from whom the biological sample was obtained is diagnosed as having CTCL or CTCL with blood involvement.

8. A method of detecting cutaneous T cell lymphoma (CTCL) or CTCL with blood involvement, comprising:
(a) analyzing nucleic acids from a biological sample for a genetic abnormality in TP53, MYC, RB1, CDKN2A, ATM, STAT3/5B, ARID1A, ZEB1, FAS, CARD11, and DNMT3A; and
(b) identifying the biological sample having one or more genetic abnormalities in TP53, MYC, RB1, CDKN2A, ATM, STAT3/5B, ARID1A, ZEB1, FAS, CARD11, and DNMT3A as a biological sample having CTCL or CTCL with blood involvement,
wherein the genetic abnormality comprises at least one deletion in at least one of TP53, RB1, CDKN2A, ATM, ARID1A, ZEB1, FAS, and DNMT3A; at least one amplification in at least one of MYC, STAT3/5B, and CARD11; or at least one deletion in at least one of TP53, RB1, CDKN2A, ATM, ARID1A, ZEB1, FAS, and DNMT3A and at least one amplification in at least one of MYC, STAT3/5B, and CARD11.

9. A method of detecting cutaneous T cell lymphoma (CTCL) or CTCL with blood involvement, comprising:
(a) analyzing nucleic acids from a biological sample for a genetic abnormality in TP53, MYC, STAT3/5B, and ARID1A; and
(b) identifying a biological sample having one or more genetic abnormalities in TP53, MYC, STAT3/5B, and ARID1A as a biological sample having CTCL or CTCL with blood involvement,
wherein the genetic abnormality comprises at least one deletion in TP53, at least one deletion in ARID1A, at least one amplification in MYC or at least one amplification in STAT3/5B.

10. The method of claim 1, wherein the biological sample comprises a blood sample.

11. The method of claim 1, wherein the biological sample comprises a tissue sample.

12. The method of claim 11, wherein the tissue sample comprises a skin node or a lymph node.

13. The method of claim 1, wherein the biological sample is enriched for CD3+ lymphocytes, CD4+ lymphocytes, and/or CD3+CD4+ lymphocytes.

14. The method of claim 13, wherein the biological sample is enriched for CD7− lymphocytes and/or CD26− lymphocytes.

15. The method of claim 1, wherein CTCL with blood involvement is leukemic cutaneous T cell lymphoma (L-CTCL), Sézary syndrome (SS), mycosis fungoides (MF), or folliculotropic mucinosis.

16. The method of claim 15, wherein the mycosis fungoides (MF) is patch/plaque mycosis fungoides, folliculotropic mycosis fungoids (F-MF), or tumor-stage mycosis fungoides.

17. The method of claim 1, wherein the nucleic acid is analyzed using a fluorescence in situ hybridization (FISH) assay, a nucleic acid sequencing assay, a single nucleotide polymorphism (SNP) array or a transcript array.

18. A probe set comprising one or more probes that binds to a nucleic acid sequence of TP53, MYC, RB1, CDKN2A, ATM, STAT3/5B, ARID1A, ZEB1, FAS, CARD11, or DNMT3A.

19. The probe set of claim 18, wherein the probe has a nucleic acid sequence provided in Table 2 or Table 3.

20. The probe set of claim 18, wherein the one or more probes comprises a detectable label.

21. A probe set comprising one or more probes that binds to a nucleic acid sequence of TP53, MYC, STAT3/5B, or ARID1A.
